(19) United States
(12) Patent Application Publication
(10) Pub. No.: US 2023/0422600 A1
(43) Pub. Date: Dec. 28, 2023

(54) DIVALENT PLATINUM COMPLEX

(71) Applicant: GUANGDONG AGLAIA OPTOELECTRONIC MATERIALS CO., LTD, Foshan, Guangdong (CN)

(72) Inventors: Xinwei WU, Foshan (CN); Huiyang LI, Foshan (CN); Wenyuan SHEN, Foshan (CN); Xiaofeng TAN, Foshan (CN); Lei DAI, Foshan (CN); Lifei CAI, Foshan (CN)

(73) Assignee: GUANGDONG AGLAIA OPTOELECTRONIC MATERIALS CO., LTD, Foshan, Guangdong (CN)

(21) Appl. No.: 18/038,679

(22) PCT Filed: Oct. 24, 2021
(86) PCT No.: PCT/CN2021/125931

§ 371 Date: May 24, 2023

(30) Foreign Application Priority Data
Dec. 21, 2020 (CN) 202011516978.5
Dec. 30, 2020 (CN) 202011618074.3

(51) Int. Cl. H10K 85/30; H10K 85/60; H10K 50/12; C07F 15/00

(52) U.S. Cl. CPC H10K 85/346; H10K 85/654; H10K 85/6572; H10K 85/615; H10K 85/6574; H10K 85/6576; H10K 50/12; C07F 15/0086

(57) ABSTRACT

The present invention relates to a bivalent platinum complex having a structure as shown in a formula (I). The complex has bright green light emission wavelengths, and can be used in the field of OLED organic electroluminescent materials. By means of the structural design, the present invention can improve the heavy atomic effect of phosphorescent materials, enhance spin-spin coupling, and achieve high-efficiency conversion of T1-S0, thereby achieving high luminous efficiency. Furthermore, a platinum complex molecule having an ONCN tetradentate ligand has the advantages of simple synthetic steps, relatively easy coordination and the like, and has may modifiable sites, which can be used for adjusting PL light emission wavelengths and thermal stability. The steric hindrance added by a carbazole group contained can effectively reduce the aggregation effect between molecules, avoid the formation of an exciplex, and further improve the color purity and the luminous efficiency.

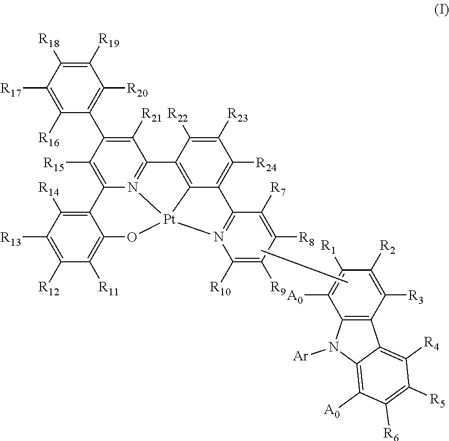

### TECHNICAL FIELD

The present invention relates to the field of OLED materials, and in particular to a bivalent platinum complex containing carbazole.

### BACKGROUND

An organic light-emitting diode (OLED) has the advantages such as self-luminous property, wide viewing angle, almost infinite high contrast, low power consumption, extremely high reaction speed and potential flexible foldability, thus having been widely concerned and studied in the past twenty years. Since the OLED was discovered by a Chinese American professor Ching W. Tang in a laboratory in 1979, it was found by S. R. Ferrest et al. in 1998 that an organic metal complex can achieve fast intersystem crossing (ISC) and long-life phosphorescent decay due to strong spin-orbit coupling (SOC). Moreover, studies have show that phosphorescent materials can make full use of energy of singlet and triplet excitons in a luminescence process, and improve the luminous efficiency of complexes, thereby achieving the internal quantum efficiency of 100% theoretically in the OLED. In particular, may studies have been carried out on Ir(I) complexes, which are luminescent materials widely used in the industry at present. However, the rare earth metal Ir has high cost and great pollution, so that application of this metal in mass production is hindered. Therefore, cheap metal complexes are required to be developed urgently. Due to excellent material stability caused by planarity, strong structure modifiability and lower metal price than iridium, a metal platinum complex has also been widely concerned in the scientific research industry in recent years, and also has a mature industrialization process. Nevertheless, simple luminescent materials having high efficiency and long service life are still required urgently in the industry at present. A platinum complex molecule having an ONCN tetradentate ligand has simple synthetic steps and may modifiable sites, and can be greatly improved.

### SUMMARY

In view of the above problems of the prior art, the present invention provides a platinum complex luminescent material having an ONCN tetradentate ligand and containing a functional group. The material can improve a heavy atomic effect of phosphorescent materials, thereby improving the utilization rate of energy of singlet and triplet excitons in a luminescence process. Through connection of the functional group at different sites, the thermal stability of a material is improved, and the possibility for further industrialization of the material is provided.

The platinum complex applied in an organic light-emitting diode shows good thermal stability, photoelectric properties and device service life.

A bivalent platinum complex has a structural formula as shown in a formula (I):

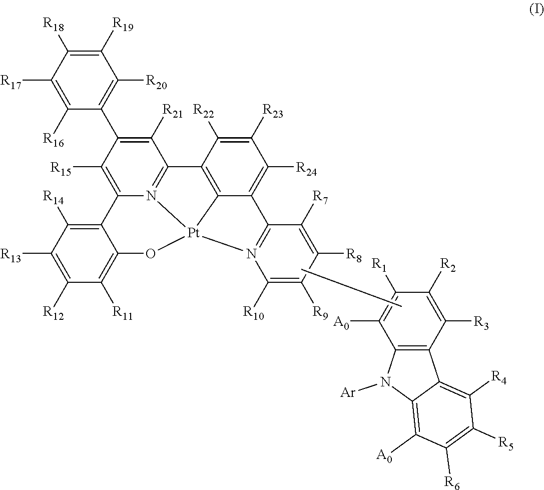

where one site of R₁-R₆ in R₁-R₂₄ is connected to one site of R₇-R₁₀ by a C—C bond, remaining sites and A₀ are independently selected from hydrogen, deuterium, halogen, substituted or unsubstituted alkyl containing 1-20 carbon atoms, substituted or unsubstituted cycloalkyl containing 3-20 ring carbon atoms, substituted or unsubstituted heteroalkyl containing 1-20 carbon atoms, substituted or unsubstituted aralkyl containing 7-30 carbon atoms, substituted or unsubstituted alkoxyl containing 1-20 carbon atoms, substituted or unsubstituted aryloxyl containing 6-30 carbon atoms, substituted or unsubstituted alkenyl containing 2-20 carbon atoms, substituted or unsubstituted aryl containing 6-30 carbon atoms, substituted or unsubstituted heteroaryl containing 3-30 carbon atoms, substituted or unsubstituted alkylsilyl containing 3-20 carbon atoms, substituted or unsubstituted arylsilyl containing 6-20 carbon atoms, substituted or unsubstituted amino containing 0-20 carbon atoms, acyl, carbonyl, carboxyl, ester group, cyano, thio, sulfinyl, sulfonyl, and phosphino, or any adjacent two of the R₁-R₂₄ are connected to each other through a covalent bond to form a ring;

Ar is selected from substituted or unsubstituted alkyl containing 1-20 carbon atoms, substituted or unsubstituted cycloalkyl containing 3-20 ring carbon atoms, substituted or unsubstituted heteroalkyl containing 1-20 carbon atoms, substituted or unsubstituted aralkyl containing 7-30 carbon atoms, substituted or unsubstituted alkoxyl containing 1-20 carbon atoms, substituted or unsubstituted aryloxyl containing 6-30 carbon atoms, substituted or unsubstituted alkenyl containing 2-20 carbon atoms, substituted or unsubstituted aryl containing 6-30 carbon atoms, substituted or unsubstituted heteroaryl containing 3-30 carbon atoms, substituted or unsubstituted alkylsilyl containing 3-20 carbon atoms, and substituted or unsubstituted arylsilyl containing 6-20 carbon atoms;
the heteroalkyl or the heteroaryl includes an N, S, or O heteroatom;
and the “substituted” refers to substitution with deuterium, halogen, amino, nitro, cyano, or C₁-C₄ alkyl.

Preferably, one site of the R₁-R₆ in the R₁-R₂₄ is connected to one site of the R₇-R₁₀ by a C—C bond, the remaining sites and the A₀ are independently selected from hydrogen, deuterium, halogen, alkoxyl containing 1-10 C atoms, cyano, styrenyl, aryloxyl, diarylamino, saturated alkyl containing 1-10 C atoms, unsaturated alkyl containing 2-8 C atoms, substituted or unsubstituted aryl containing 5-20 C atoms, and substituted or unsubstituted heteroaryl containing 5-20 C atoms, or any adjacent two of the R₁-R₂₄ are connected to each other through a covalent bond to form a ring.

The Ar is selected from a substituted or unsubstituted o-aryl derivative or o-heteroaryl derivative containing 6-30 carbon atoms, and a substituted or unsubstituted biaryl derivative or biheteroaryl derivative containing 3-30 carbon atoms.

Further preferably,
one site of the R₁-R₆ is connected to one site of the R₇-R₁₀ by a C—C bond, the remaining sites in the R₁-R₂₄ are hydrogen except that R₁₇ and R₁₉ are a C₁-C₄ group, and the A₀ is hydrogen.

The Ar is selected from substituted or unsubstituted aryl containing 5-30 carbon atoms, heteroaryl, and benzoheteroaryl.

Preferably, the R₁₇ and the R₁₉ are isobutyl; the Ar is selected from substituted or unsubstituted five-membered or six-membered heteroaryl containing phenyl, and benzoheteroaryl; the heteroaryl includes an N or O heteroatom; and the “substituted” refers to substitution with deuterium, halogen, or C₁-C₄ alkyl.

The platinum metal complex of the present invention is listed below, but is not limited to the structures listed:

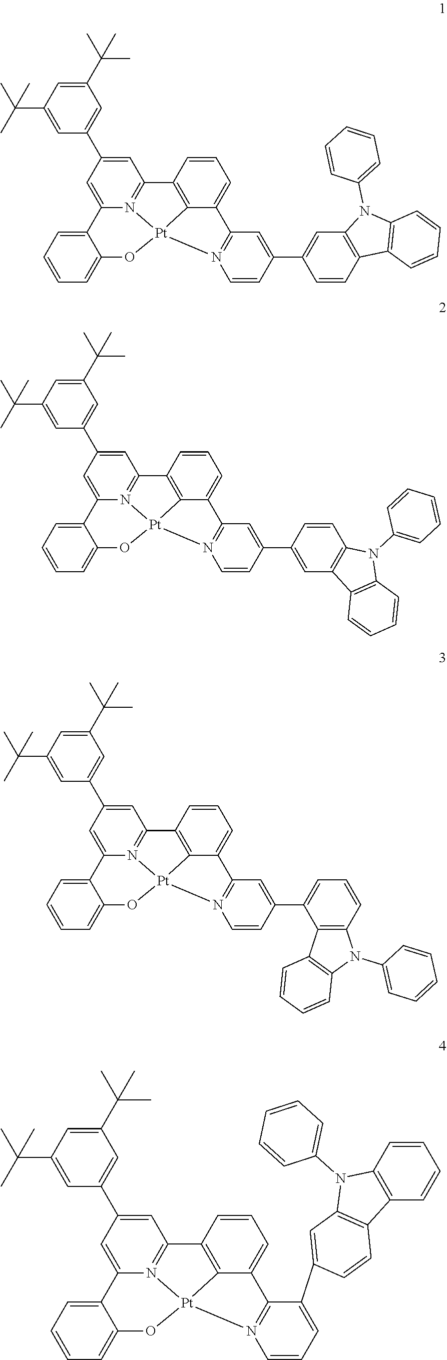

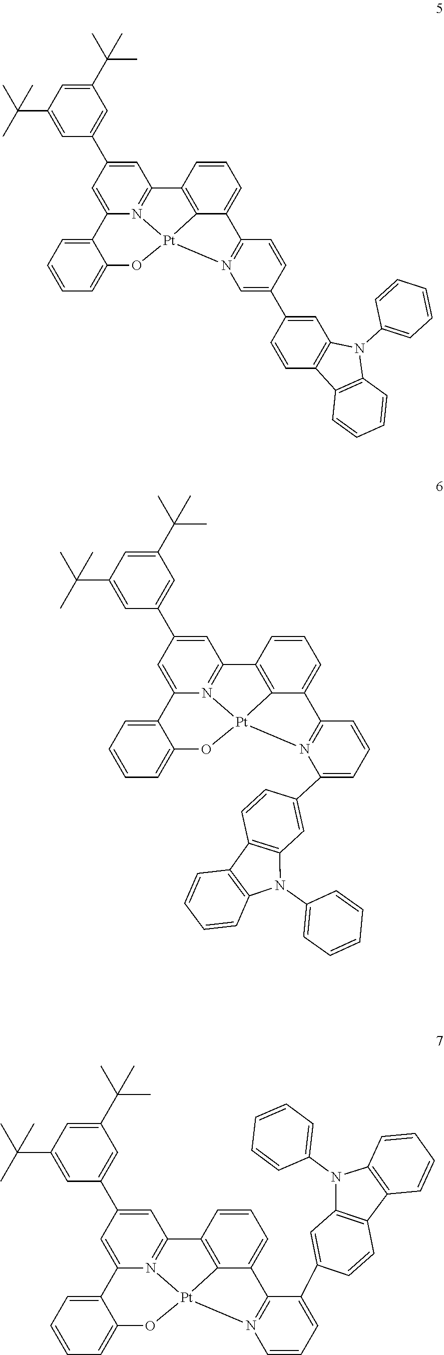

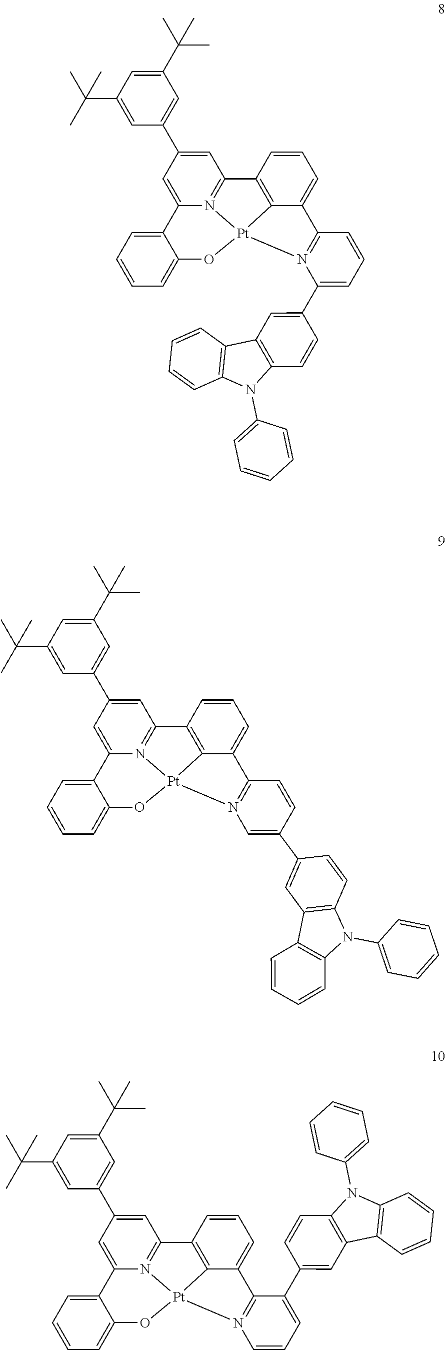

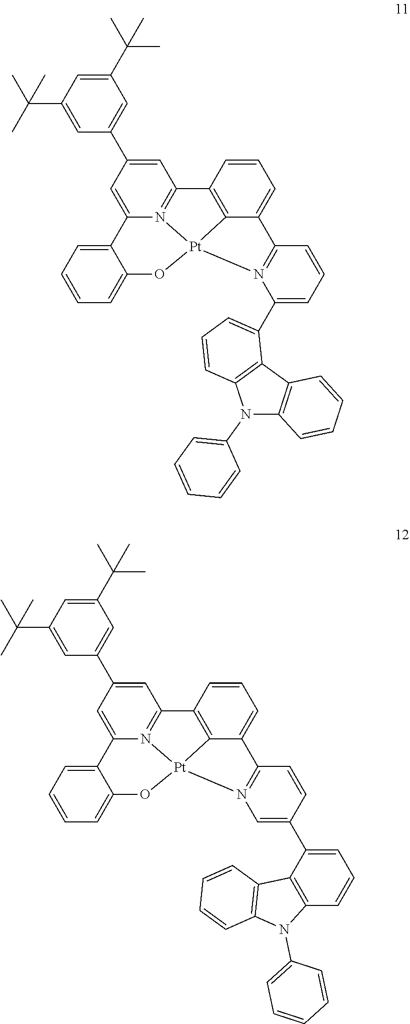

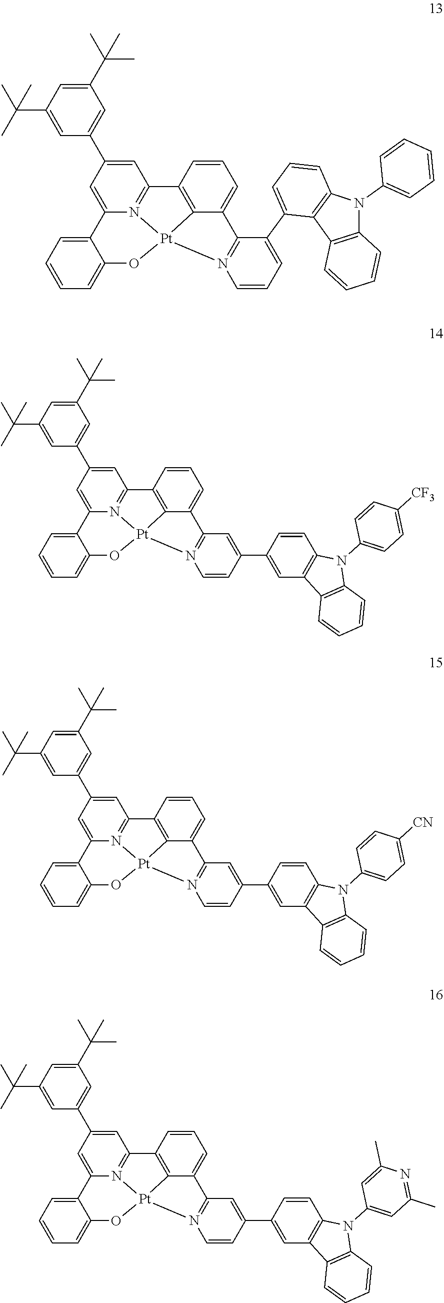

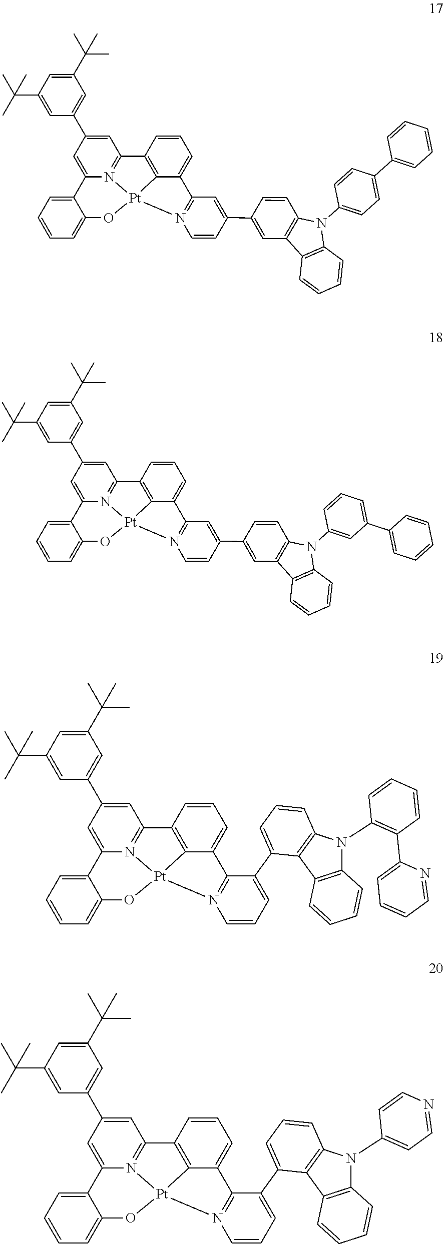

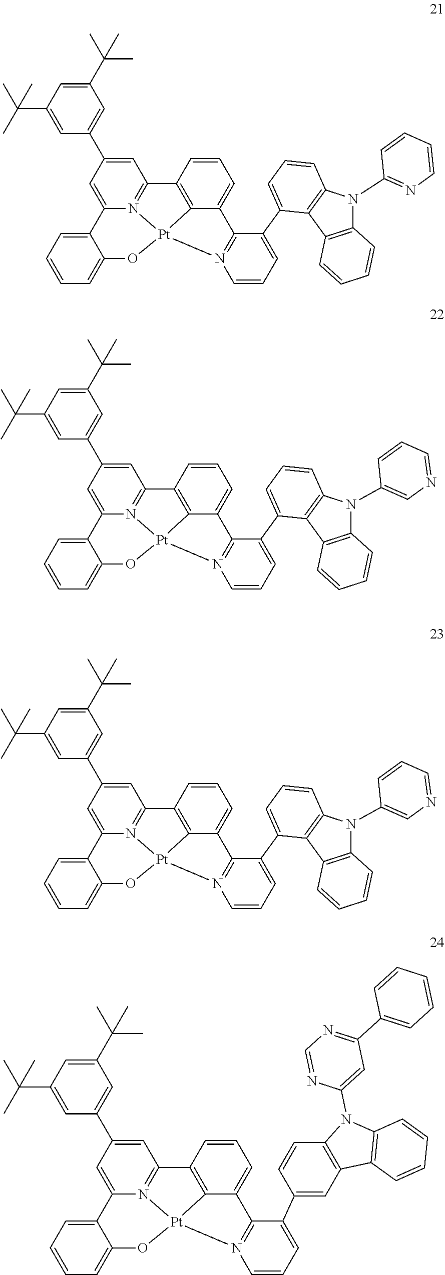

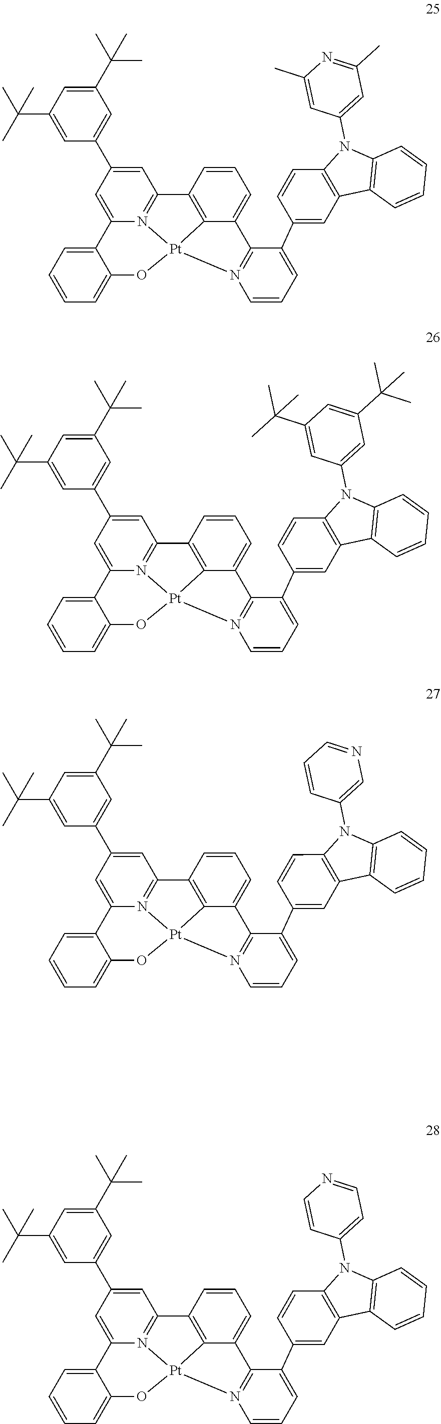

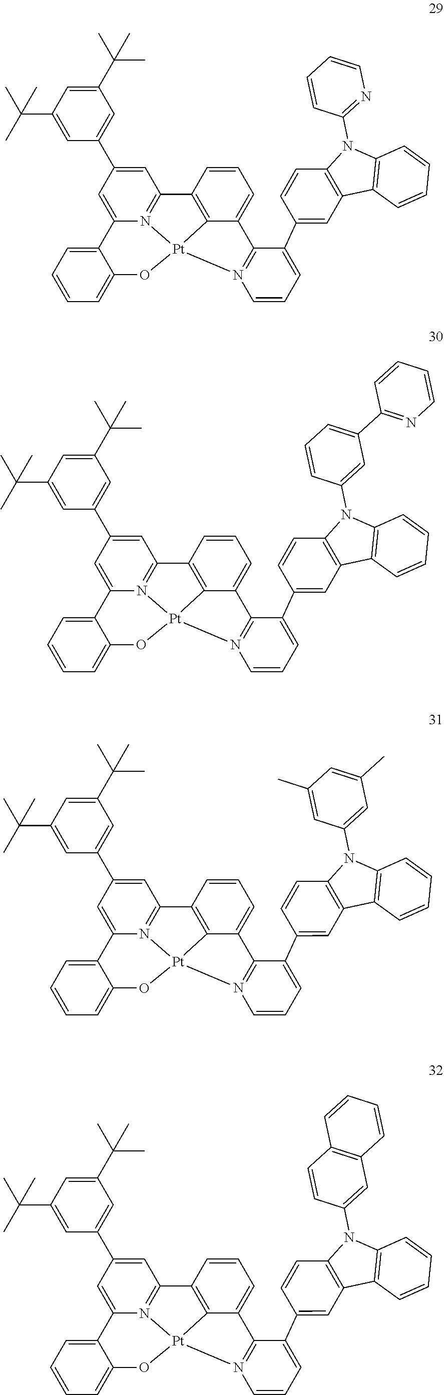

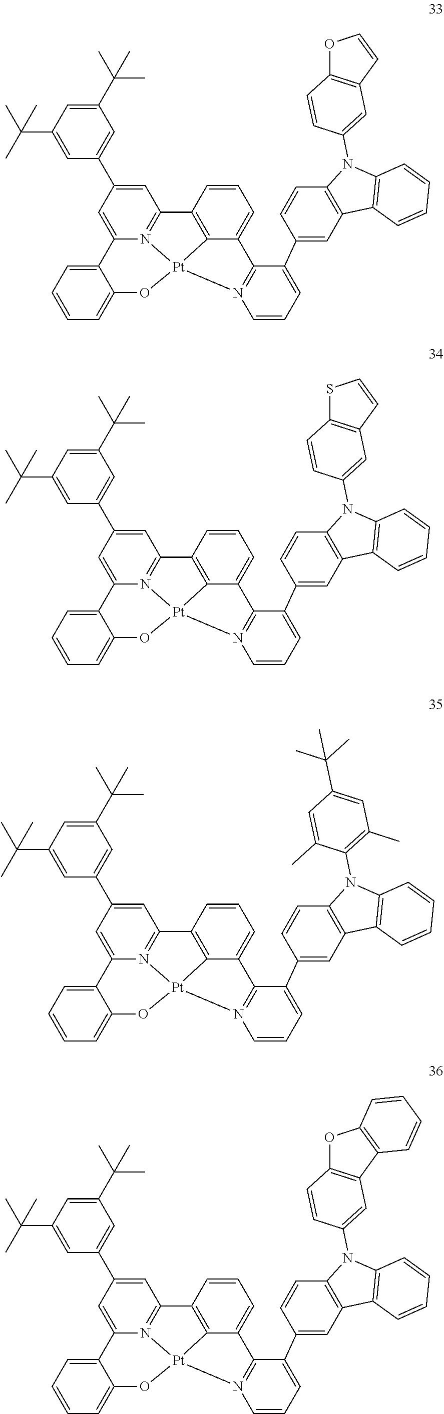

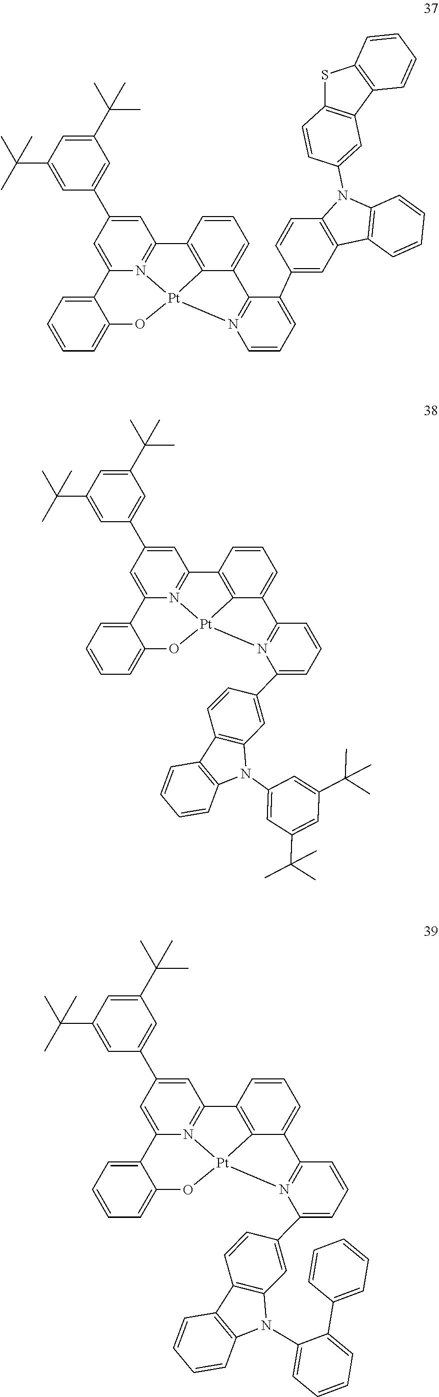

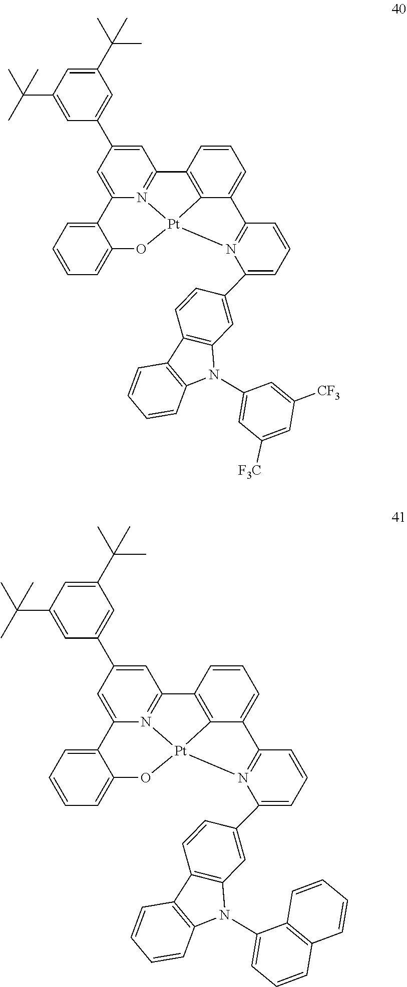

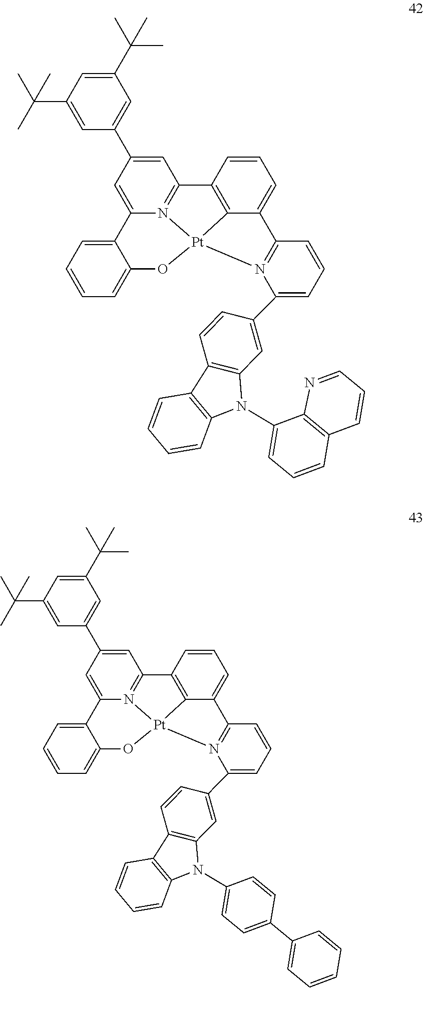

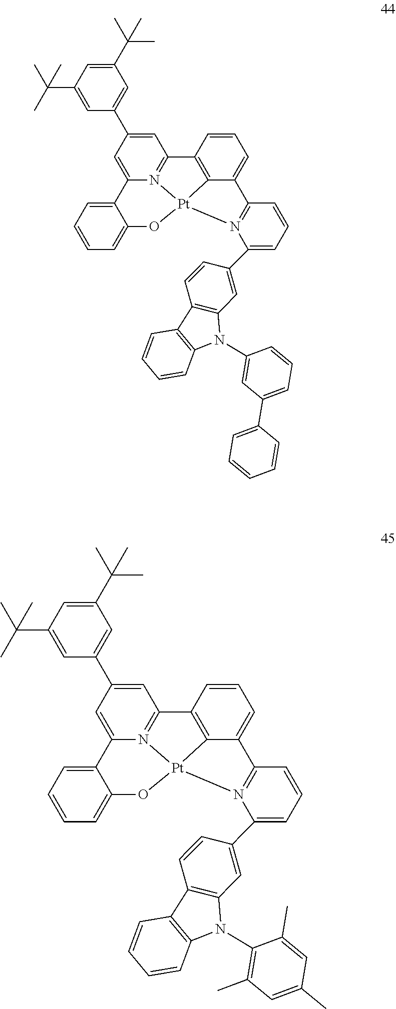

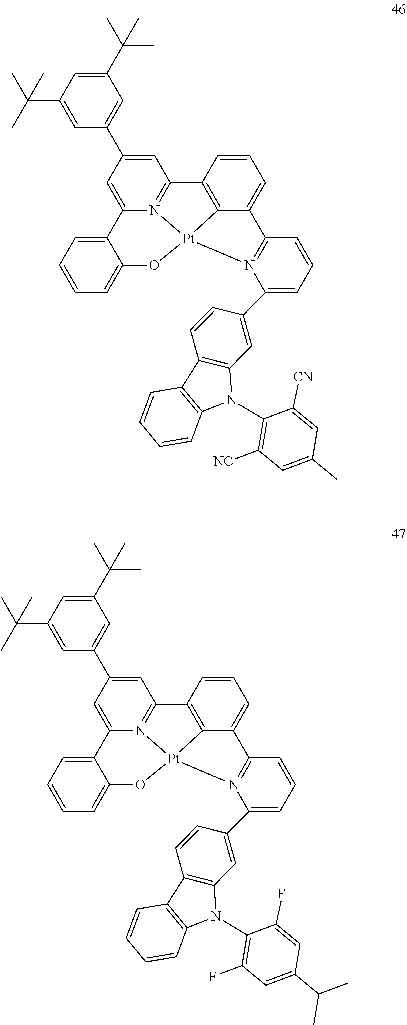

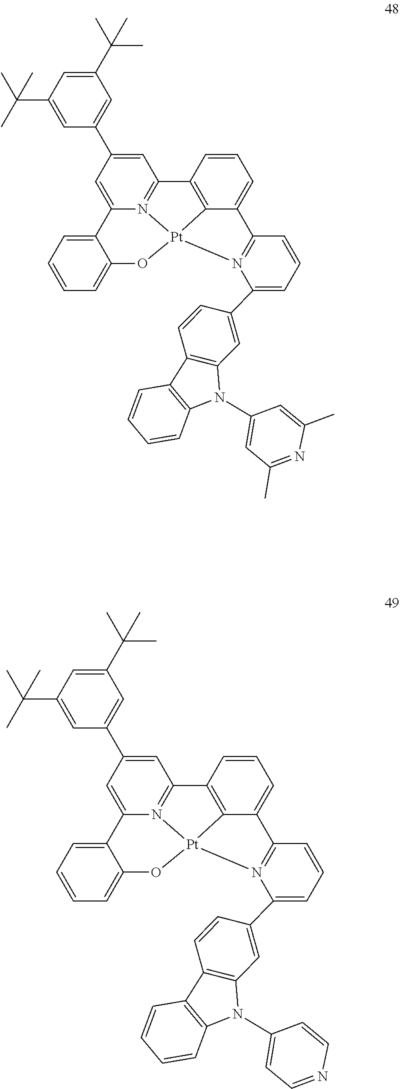

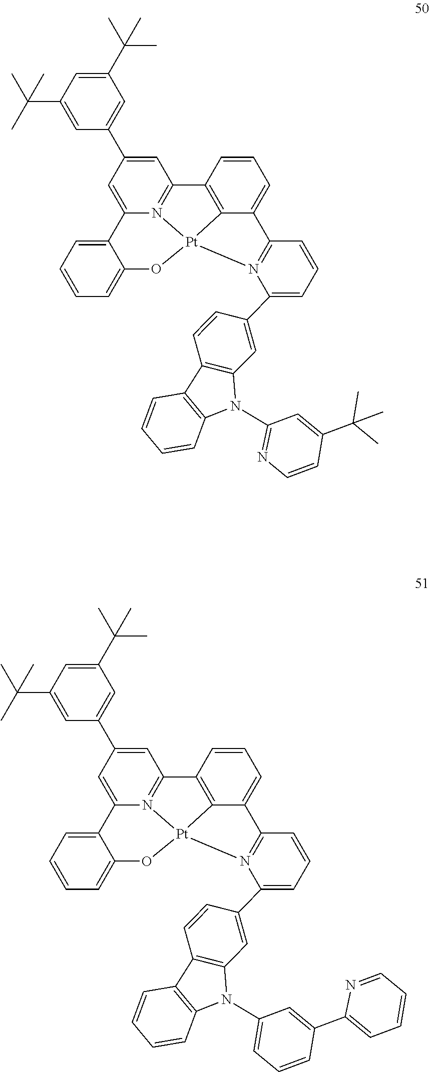

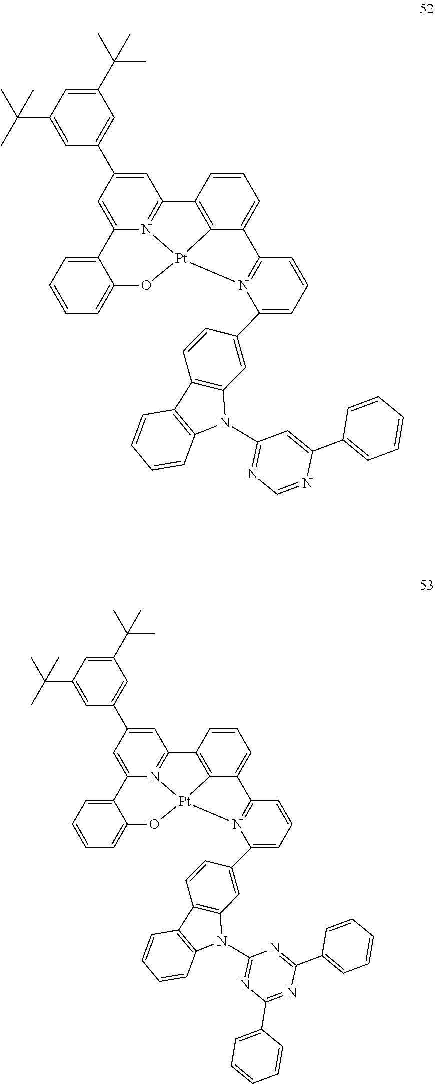

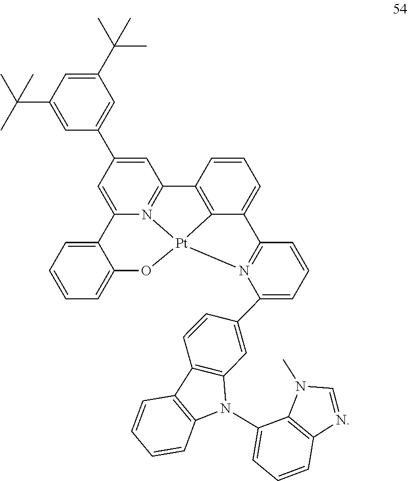

A precursor of the complex has a structure as shown in the following formula (II):

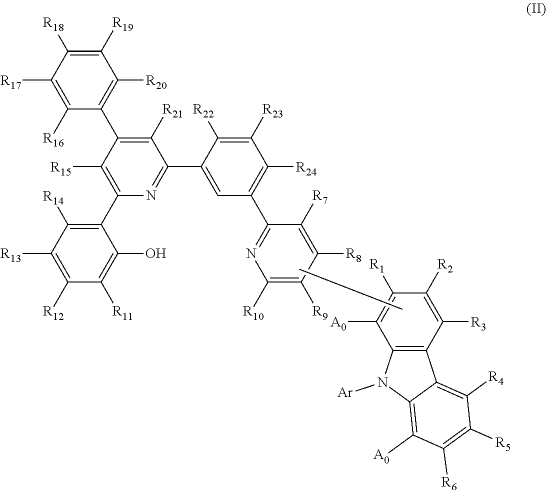

where R₁-R₂₄, A₀ and Ar are defined the same as above.

The bivalent platinum complex of the present invention is applied as a phosphorescent doping material for a light-emitting layer in an OLED.

By means of the structural design, the present invention improves the stability of a material, the efficiency of a device and the service life.

The compound has simple synthetic steps, and can easily obtain a mature process.

The structure has many modifiable sites, and the steric hindrance added by a carbazole group contained can effectively reduce the aggregation effect between molecules.

Through connection of the functional group at different sites, the compound can improve the spatial configuration of a molecule, and improve the color purity and the luminous efficiency.

The bivalent platinum complex of the present invention has bright green light emission wavelengths, and can be used in the field of OLED organic electroluminescent materials. By means of the structural design, the present invention can improve the heavy atomic effect of phosphorescent materials, enhance spin-spin coupling, and achieve high-efficiency conversion of T1-S0, thereby achieving high luminous efficiency. Moreover, a platinum complex molecule having an ONCN tetradentate ligand has the advantages of simple synthetic steps, relatively easy coordination and the like, and has may modifiable sites, which can be used for adjusting PL light emission wavelengths and thermal stability. The steric hindrance added by a carbazole group contained can effectively reduce the aggregation effect between molecules, avoid the formation of an exciplex, and further improve the color purity and the luminous efficiency.

The organic metal complex in the present invention has high fluorescence quantum efficiency, good thermal stability and low quenching constant, and can be used for manufacturing a green light OLED device having high luminous efficiency.

### BRIEF DESCRIPTION OF DRAWINGS

FIG. 1 is a structural diagram of a device of the present invention.

### DETAILED DESCRIPTION OF EMBODIMENTS

The present invention is further limited below in conjunction with attached drawings and embodiments.

All raw materials used below are commercially available.

### EXAMPLE 1

Synthesis of a compound having a structure 1 (1a was prepared with reference to CN110872325A, and 1d is an ordered material)

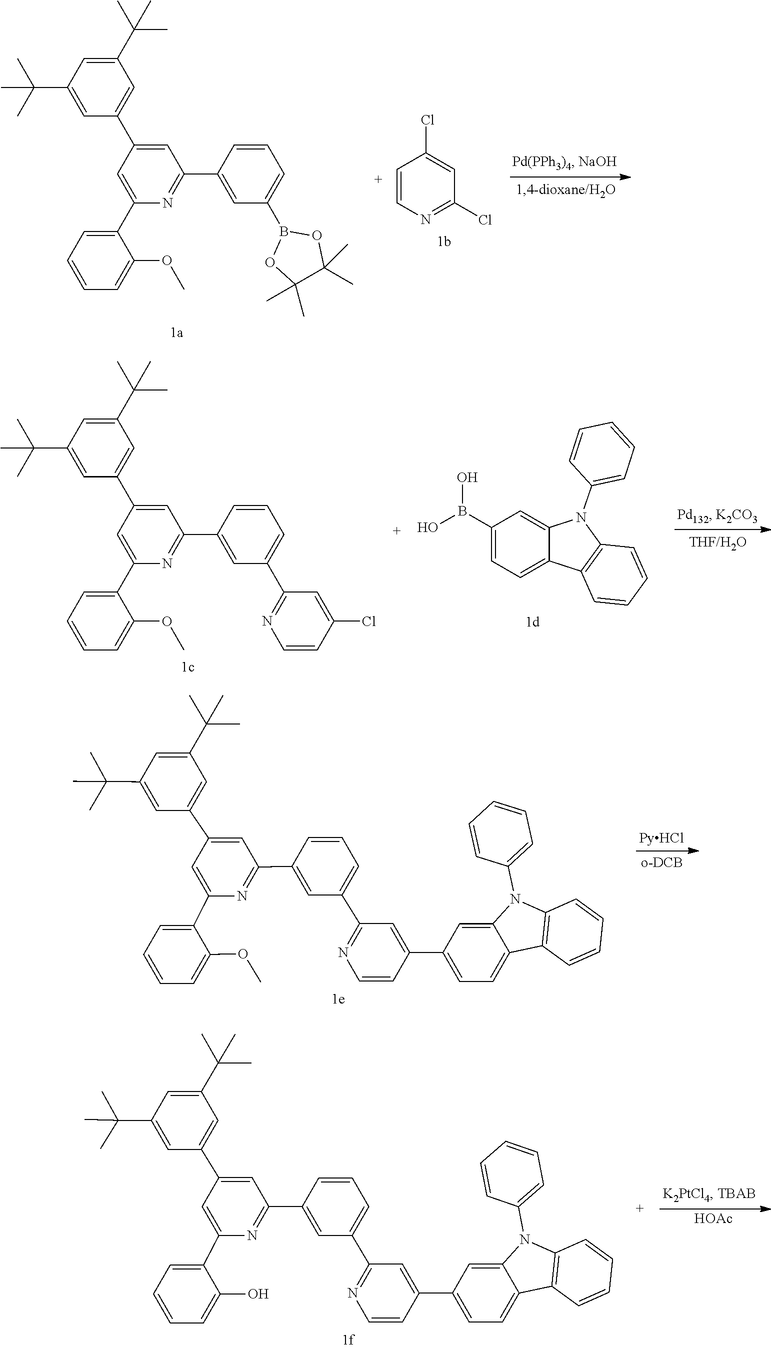

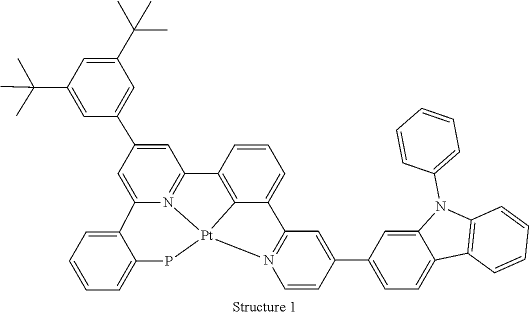

### Synthesis of an Intermediate 1c

1a (60.0 g, 101 mmol), 1b (30.0 g, 202 mmol), Pd(PPh₃)₄ (5.90 g, 5 mmol), NaOH (8.2 mg, 202 mmol), and dioxane/water (1.2 L/240 ml) were put into a 2 L three-mouth flask, and stirred for a reaction at 55° C. for 12 h under the protection of nitrogen. After the reaction was completed, most of a solvent was spin-dried, water was added, and extraction was conducted with DCM for 2 times. Then, spin-drying was conducted with silica gel, and treatment was conducted with a silica gel column (with a mixture of Hex and EA at a ratio of 10:1). 58 g of a white solid was obtained. Hydrogen spectrum data are as follows:

¹H NMR (400 MHz, CDCl₃) δ8.69 (s, 1H), 8.61 (d, J=5.3 Hz, 1H), 8.23 (d, J=7.8 Hz, 1H), 8.12-8.01 (m, 3H), 7.91 (d, J=1.4 Hz, 1H), 7.85 (d, J=1.7 Hz, 1H), 7.62 (s, 1H), 7.55 (s, 3H), 7.47-7.39 (m, 1H), 7.30-7.26 (m, 1H), 7.16 (s, 1H), 7.06 (d, J=8.1 Hz, 1H), 3.92 (s, 3H), 1.41 (s, 18H).

### Synthesis of an Intermediate 1e

The 1c (8.0 g, 19.9 mmol), 1d (6.14 g, 21.4 mmol), Pd₁₃₂ (0.303 g, 0.42 mmol), K₂CO₃ (5.9 g, 42.7 mmol), and THF/water (80 ml/16 ml) were put into a 250 ml one-mouth flask, and subjected to a reaction at 70° C. for 12 h under the protection of nitrogen. After the reaction was completed, most of a solvent was spin-dried, water was added, and extraction was conducted with EA for 2 times. Then, spin-drying was conducted with silica gel, and treatment was conducted with a silica gel column (with a mixture of Hex and EA at a ratio of 6:1) to obtain 9.7 g of a white solid. Hydrogen spectrum data are as follows:

¹H NMR (400 MHz, CDCl₃) δ8.76 (d, J=4.1 Hz, 2H), 8.34-8.23 (m, 2H), 8.20 (d, J=7.8 Hz, 1H), 8.13 (d, J=7.8 Hz, 1H), 8.10-8.01 (m, 3H), 7.96 (s, 1H), 7.73-7.55 (m, 10H), 7.55-7.38 (m, 5H), 7.34 (t, J=6.4 Hz, 1H), 7.15 (t, J=7.5 Hz, 1H), 7.07 (d, J=8.2 Hz, 1H), 3.93 (s, 3H), 1.44 (s, 18H).

### Synthesis of an Intermediate 1f

The 1e (8.5 g, 11.06 mmol), pyridine hydrochloride (90 g), and 9 mL of o-dichlorobenzene were put into a 500 ml one-mouth flask, and subjected to a reaction at 200° C. for 6 h under the protection of nitrogen. After the reaction was completed, extraction was conducted with dichloromethane for two times, spin-drying was conducted, and treatment was conducted with a silica gel column (with a mixture of Hex and EA at a ratio of 6:1). Then, a resulting crude product was subjected to beating with methanol. 8.3 g of a light yellow solid was obtained. Hydrogen spectrum data are as follows:

¹H NMR (400 MHz, CDCl₃) δ8.74 (d, J=4.8 Hz, 1H), 8.64 (s, 1H), 8.25 (d, J=8.1 Hz, 1H), 8.17 (dd, J=12.1, 8.0 Hz, 2H), 8.07 (t, J=9.5 Hz, 3H), 7.95 (d, J=8.1 Hz, 1H), 7.92 (d, J=1.1 Hz, 1H), 7.73-7.64 (m, 3H), 7.64-7.57 (m, 5H), 7.56-7.50 (m, 3H), 7.50-7.39 (m, 3H), 7.39-7.28 (m, 2H), 7.12-7.04 (m, 1H), 6.97 (t, J=7.0 Hz, 1H), 1.43 (s, 18H).

### Synthesis of an End-Product Compound Having a Structure 1

The 1f (2.0 g, 2.65 mmol), K₂PtCl₄ (1.32 g, 3.18 mmol), TBAB (85 mg, 0.265 mmol), and acetic acid (200 mL) were put into a 500 ml one-mouth flask, and subjected to a reaction at 130° C. for 48 h under the protection of nitrogen. After the reaction was completed, an excessive amount of deionized water was added to precipitate out a solid. Then suction filtration was conducted, and a solid was dissolved in dichloromethane, spin-dried, and subjected to treatment with a silica gel column (with DCM). 1.6 g of a yellow solid was obtained. Hydrogen spectrum data are as follows:

¹H NMR (400 MHz, CDCl₃) δ8.89 (d, J=5.9 Hz, 1H), 8.21-8.01 (m, 3H), 7.88 (d, J=7.7 Hz, 1H), 7.74-7.47 (m, 12H), 7.47-7.34 (m, 3H), 7.34-7.28 (m, 3H), 7.24 (d, J=5.6 Hz, 2H), 7.07 (t, J=7.5 Hz, 1H), 6.52 (t, J=7.4 Hz, 1H), 1.45 (s, 18H).

### EXAMPLE 2: SYNTHESIS OF A COMPOUND HAVING A STRUCTURE 26 (26a IS AN ORDERED MATERIAL)

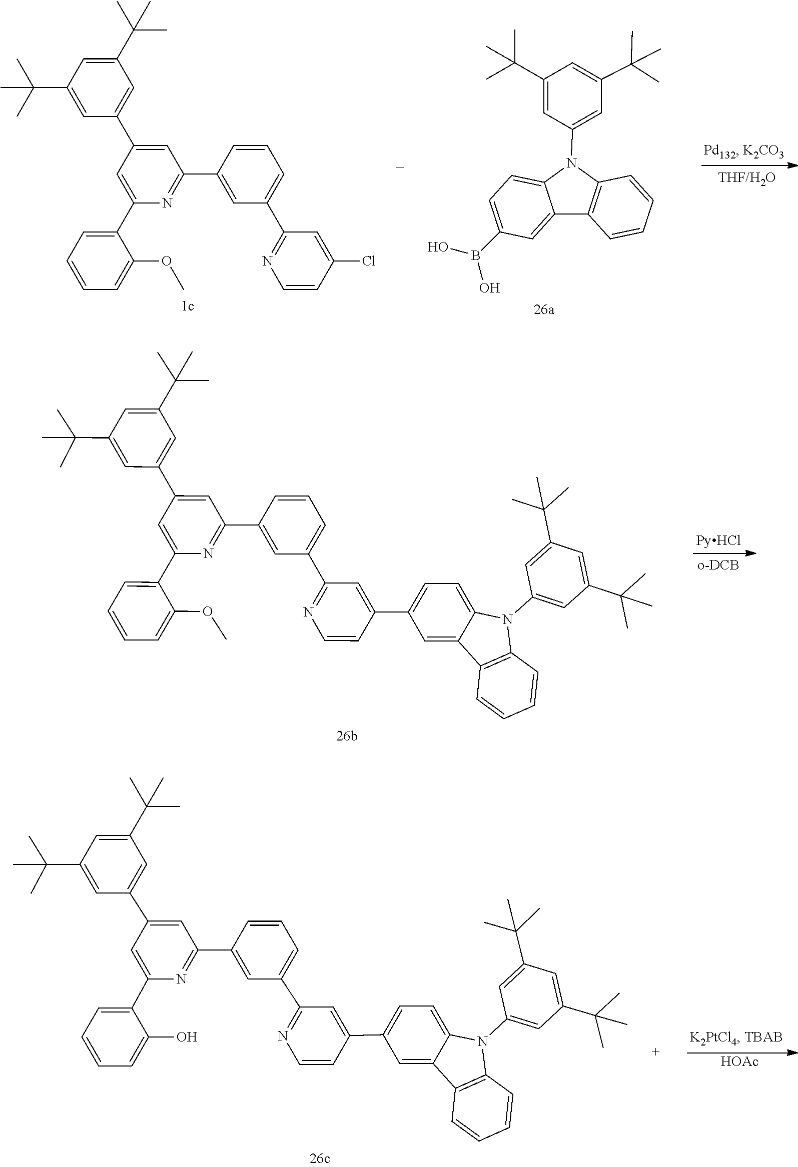

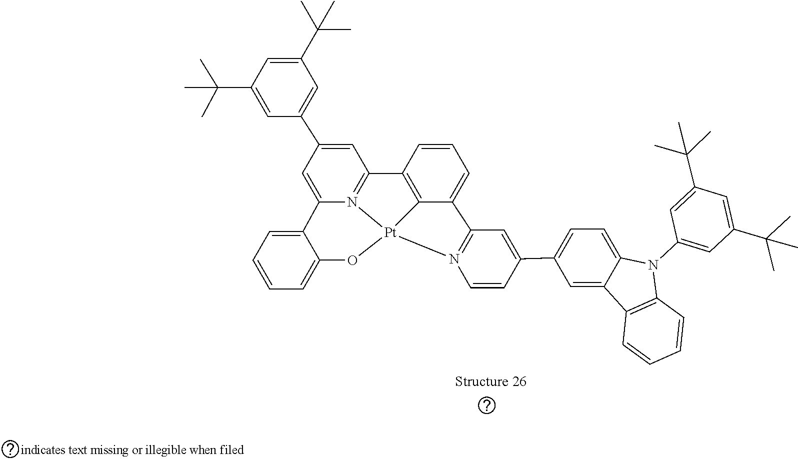

### Synthesis of an Intermediate 26b

The 1c (8.0 g, 19.9 mmol), 26a (8.5 g, 21.4 mmol), Pd₁₃₂ (0.303 g, 0.42 mmol), K₂CO₃ (5.9 g, 42.7 mmol), and THF/water (80 ml/16 ml) were put into a 250 ml one-mouth flask, and subjected to a reaction at 70° C. for 12 h under the protection of nitrogen. After the reaction was completed, most of a solvent was spin-dried, water was added, and extraction was conducted with EA for 2 times. Then, spin-drying was conducted with silica gel, and treatment was conducted with a silica gel column (with a mixture of Hex and EA at a ratio of 6:1) to obtain 11.08 g of a white solid. Hydrogen spectrum data are as follows:

¹H NMR (400 MHz, Chloroform-d) δ8.67 (d, J=4.5 Hz, 1H), 8.22-8.17 (m, 3H), 8.17-8.08 (m, 3H), 7.95-7.86 (m, 4H), 7.72 (dd, J=6.8, 2.4 Hz, 1H), 7.69-7.59 (m, 2H), 7.53-7.27 (m, 10H), 7.15 (ddd, J=8.7, 7.5, 1.2 Hz, 1H), 6.90 (dd, J=7.7, 1.2 Hz, 1H), 3.90 (s, 3H), 1.35 (s, 36H).

### Synthesis of an Intermediate 26c

The 26b (9.74 g, 11.06 mmol), pyridine hydrochloride (90 g), and 9 mL of o-dichlorobenzene were put into a 500 ml one-mouth flask, and subjected to a reaction at 200° C. for 6 h under the protection of nitrogen. After the reaction was completed, extraction was conducted with dichloromethane for two times, spin-drying was conducted, and treatment was conducted with a silica gel column (with a mixture of Hex and EA at a ratio of 6:1). Then, a resulting crude product was subjected to beating with methanol. 8.3 g of a light yellow solid was obtained. Hydrogen spectrum data are as follows:

¹H NMR (400 MHz, Chloroform-d) δ9.18-9.11 (m, 1H), 8.26-8.09 (m, 5H), 7.94 (dd, J=8.2, 1.2 Hz, 1H), 7.74 (d, J=2.1 Hz, 1H), 7.68-7.62 (m, 3H), 7.60-7.52 (m, 2H), 7.51-7.44 (m, 3H), 7.40-7.26 (m, 9H), 7.17 (d, J=6.7 Hz, 2H), 7.09 (ddd, J=8.4, 7.5, 1.2 Hz, 1H), 1.36 (d, J=2.9 Hz, 38H).

### Synthesis of an End-Product Compound Having a Structure 26

The 26c (2.3 g, 2.65 mmol), K₂ PtCl₄ (1.32 g, 3.18 mmol), TBAB (85 mg, 0.265 mmol), and acetic acid (200 mL) were put into a 500 ml one-mouth flask, and subjected to a reaction at 130° C. for 48 h under the protection of nitrogen. After the reaction was completed, an excessive amount of deionized water was added to precipitate out a solid. Then suction filtration was conducted, and a solid was dissolved in dichloromethane, spin-dried, and subjected to treatment with a silica gel column (with DCM). 1.8 g of a yellow solid was obtained. Hydrogen spectrum data are as follows:

¹H NMR (400 MHz, CDCl₃) δ8.89 (d, J=5.9 Hz, 1H), 8.21-8.01 (m, 3H), 7.88 (d, J=7.7 Hz, 1H), 7.74-7.47 (m, 12H), 7.47-7.34 (m, 3H), 7.34-7.28 (m, 3H), 7.24 (d, J=5.6 Hz, 2H), 7.07 (t, J=7.5 Hz, 1H), 6.52 (t, J=7.4 Hz, 1H), 1.45 (s, 18H).

### EXAMPLE 3: SYNTHESIS OF A COMPOUND HAVING A STRUCTURE 36 (36a IS AN ORDERED MATERIAL)

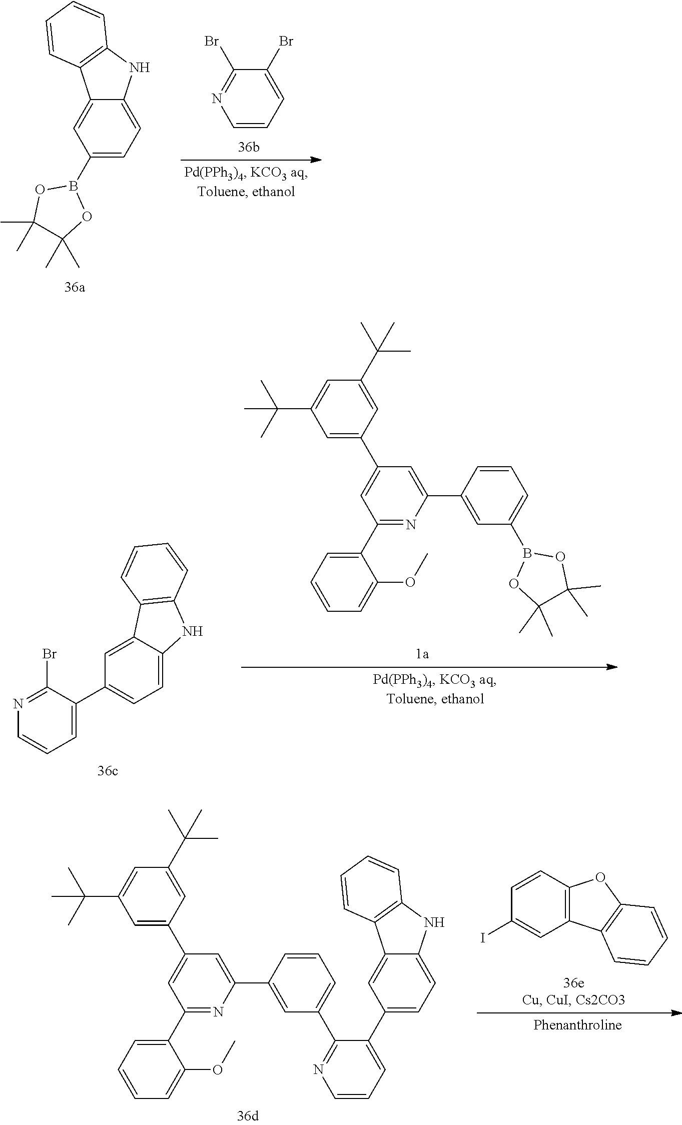 [SMILES: CC1(C)OB(c2ccc3[nH]c4ccccc4c3c2)OC1(C)C ; Brc1cccnc1Br ; Brc1ncccc1-c1ccc2[nH]c3ccccc3c2c1 ; COc1ccccc1-c1cc(-c2cc(C(C)(C)C)cc(C(C)(C)C)c2)cc(-c2cccc(B3OC(C)(C)C(C)(C)O3)c2)n1 ; COc1ccccc1-c1cc(-c2cc(C(C)(C)C)cc(C(C)(C)C)c2)cc(-c2cccc(-c3ncccc3-c3ccc4[nH]c5ccccc5c4c3)c2)n1 ; Ic1ccc2oc3ccccc3c2c1]

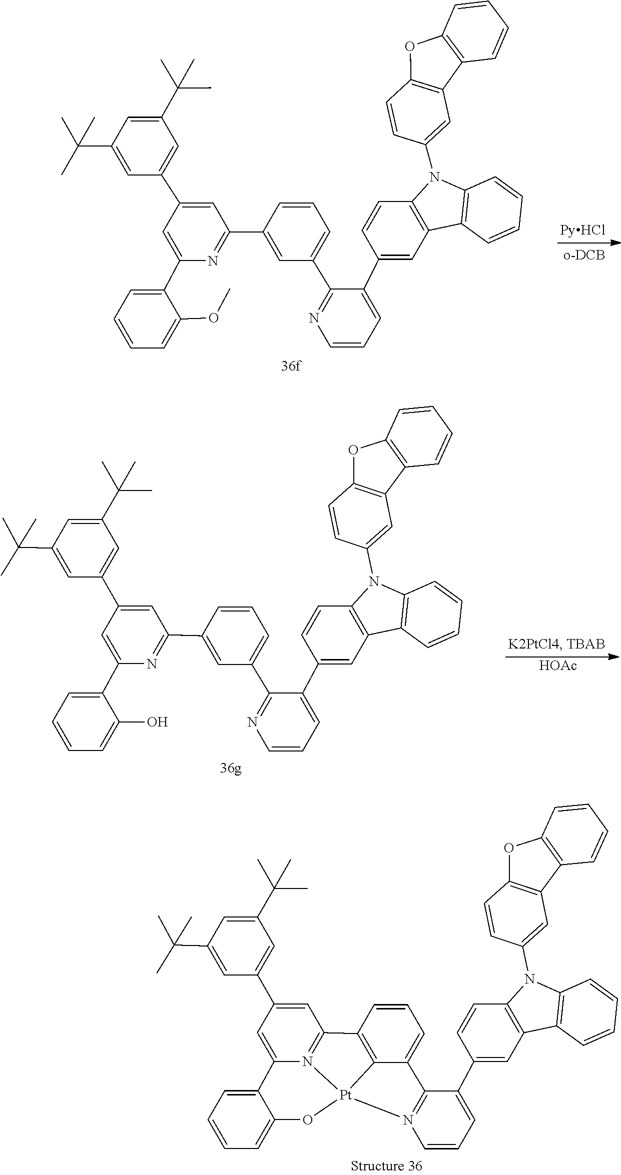

### Synthesis of an Intermediate 36c

36a (14.7 g, 50 mmol), 36b (11.8 g, 50 mmol), toluene (120 mL), ethanol (60 mL), and a 2 mol/L potassium carbonate solution (60 mL) were added into a 500 ml dry double-neck flask, and subjected to ultrasonic treatment for 5-10 min. Then, nitrogen was rapidly stirred for 5 min, tetra(triphenylphosphine)palladium (1.8 g, 1.5 mmol) as a catalyst was rapidly added, and a large amount of the nitrogen was introduced for 10 min. A resulting mixture was heated to 100° C., and stirred for 12 h. Then, during treatment, extraction was conducted first, spin-drying was conducted, and column chromatography was conducted with petroleum ether and dichloromethane to obtain 14.5 g of a white solid product with a yield of 90%. Hydrogen spectrum data are as follows:

¹H NMR (400 MHz, Chloroform-d) δ9.54 (s, 1H), 8.37 (dd, J=4.0, 2.2 Hz, 1H), 8.17-8.10 (m, 2H), 7.94 (dd, J=7.5, 2.2 Hz, 1H), 7.56 (s, 1H), 7.55-7.48 (m, 2H), 7.46 (dd, J=7.5, 3.9 Hz, 1H), 7.34 (td, J=7.4, 1.3 Hz, 1H), 7.28-7.19 (m, 1H).

### Synthesis of an Intermediate 36d

The 36c (14.5 g, 45 mmol), 1a (28.7 g, 50 mmol), toluene (120 mL), ethanol (60 mL), and a 2 mol/L potassium carbonate solution (60 mL) were added into a 500 ml dry double-neck flask, and subjected to ultrasonic treatment for 5-10 min. Then, nitrogen was rapidly stirred for 5 min, tetra(triphenylphosphine)palladium (1.8 g, 1.5 mmol) as a catalyst was rapidly added, and a large amount of the nitrogen was introduced for 10 min. A resulting mixture was heated to 100° C., and stirred for 12 h. Then, during treatment, extraction was conducted first, spin-drying was conducted, and column chromatography was conducted with petroleum ether and dichloromethane to obtain g of a white solid product with a yield of 78%. Hydrogen spectrum data are as follows:

¹H NMR (400 MHz, Chloroform-d) δ9.54 (s, 1H), 8.71 (dd, J=4.1, 2.2 Hz, 1H), 8.19 (t, J=2.0 Hz, 1H), 8.17-8.08 (m, 2H), 8.02 (dd, J=1.9, 0.7 Hz, 1H), 7.96-7.87 (m, 4H), 7.80 (ddd, J=8.5, 1.9, 1.2 Hz, 1H), 7.67 (t, J=8.6 Hz, 1H), 7.57 (d, J=8.3 Hz, 1H), 7.54-7.48 (m, 2H), 7.47-7.32 (m, 7H), 7.24 (ddd, J=7.9, 7.3, 1.6 Hz, 1H), 7.15 (ddd, J=8.7, 7.5, 1.2 Hz, 1H), 6.90 (dd, J=7.7, 1.2 Hz, 1H), 3.90 (s, 3H), 1.35 (s, 18H).

### Synthesis of an Intermediate 36f

The 36d (5.0 g, 1.0 eq), 36e (5.7 g, 3.0 eq), Cu (230 mg, 0.5 eq), CuI (688 mg, 0.5 eq), 1,10-phenanthroline (1.30 g, 1.0 eq), and cesium carbonate (7.06 g, 3.0 eq) were added into a 250 ml three-mouth flask, and subjected to a reaction at an oil bath temperature of 160° C. for 3 d under the protection of nitrogen with 100 ml of anhydrous xylene as a reaction solvent. After the reaction was completed, cooling was conducted to room temperature. Then, a resulting reaction solution was directly subjected to suction filtration with EA as an eluent to remove an inorganic salt, and chromatography was conducted with a silica gel column (with a mixture of hex and EA at a ratio of 8:1 as a chromatography solution) to obtain 3.9 g of a yellow fluorescent product point by collection. Hydrogen spectrum data are as follows: ¹H NMR (400 MHz, Chloroform-d) δ8.71 (dd, J=4.1, 2.2 Hz, 1H), 8.24 (dd, J=1.9, 0.7 Hz, 1H), 8.22-8.08 (m, 5H), 8.02 (ddt, J=8.9, 1.3, 0.5 Hz, 1H), 7.97-7.85 (m, 5H), 7.84-7.77 (m, 2H), 7.71-7.62 (m, 4H), 7.59-7.53 (m, 1H), 7.53-7.27 (m, 13H), 7.15 (ddd, J=8.7, 7.5, 1.2 Hz, 1H), 6.90 (dd, J=7.7, 1.2 Hz, 1H), 3.90 (s, 3H), 1.35 (s, 18H).

### Synthesis of an Intermediate 36g

The 36f (3.15 g), pyridine hydrochloride (30.0 g), and o-dichlorobenzene (3.0 ml) were added into a 100 ml one-mouth flask, and subjected to a reaction at an oil bath temperature of 200° C. for 8 h under the protection of nitrogen. After the reaction was completed, cooling was conducted to room temperature. Dissolution was conducted with a large amount of water, and extraction was conducted with DCM for three times. Then, an organic phase was spin-dried and subjected to chromatography with a silica gel column (with a mixture of hex and EA at a ratio of 10:1 as a chromatography solution) to obtain 2.8 g of a light yellow product. Hydrogen spectrum data are as follows: ¹H NMR (400 MHz, Chloroform-d) δ8.71 (dd, J=4.1, 2.2 Hz, 1H), 8.24 (dd, J=1.9, 0.7 Hz, 1H), 8.21-8.08 (m, 5H), 8.06-7.97 (m, 2H), 7.97-7.77 (m, 6H), 7.71-7.62 (m, 4H), 7.59-7.53 (m, 1H), 7.53-7.44 (m, 5H), 7.44-7.20 (m, 8H), 7.03-6.94 (m, 2H), 1.35 (s, 18H).

### Synthesis of an End-Product Compound Having a Structure 36

The 36g (105 mg, 0.124 mmol), K₂ PtCl₄ (70 mg, 0.167 mmol), 18-crown-6-ether (6 mg, 0.012 mmol), and acetic acid (5 mL) were put into a 250 ml one-mouth flask, and subjected to a reaction at 130 ° C. for 48 h under the protection of nitrogen. After the reaction was completed, an excessive amount of deionized water was added to precipitate out a solid. Then suction filtration was conducted, and a solid was dissolved in dichloromethane, spin-dried, and subjected to treatment with a silica gel column (with a mixture of Hex, DCM and EA at a ratio of 20:20:1). After the treatment with the column, a resulting product was subjected to recrystallization with a mixture of dichloromethane and n-hexane at a ratio of 1:4. 85 mg of a red solid was obtained. Hydrogen spectrum data are as follows: ¹H NMR (400 MHz, Chloroform-d) δ8.84 (dd, J=5.4, 2.3 Hz, 1H), 8.21 (d, J=1.8 Hz, 1H), 8.17-7.99 (m, 5H), 7.94 (dd, J=8.1, 1.2 Hz, 1H), 7.81 (t, J=1.2 Hz, 1H), 7.76-7.62 (m, 6H), 7.58-7.43 (m, 12H), 7.42-7.26 (m, 8H), 7.25-7.15 (m, 2H), 7.09 (ddd, J=8.5, 7.5, 1.2 Hz, 1H), 1.35 (s, 18H).

### EXAMPLE 4: SYNTHESIS OF A COMPOUND HAVING A STRUCTURE 53 (53a IS AN ORDERED MATERIAL)

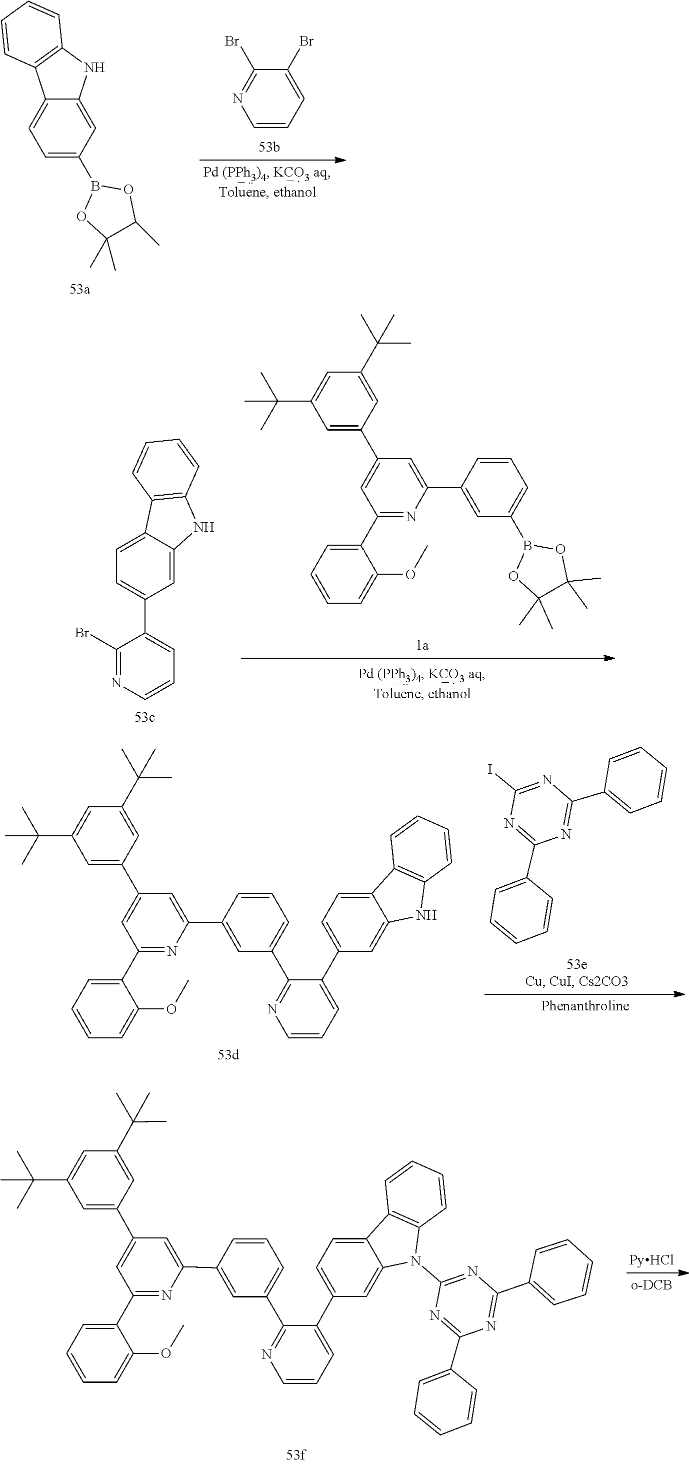

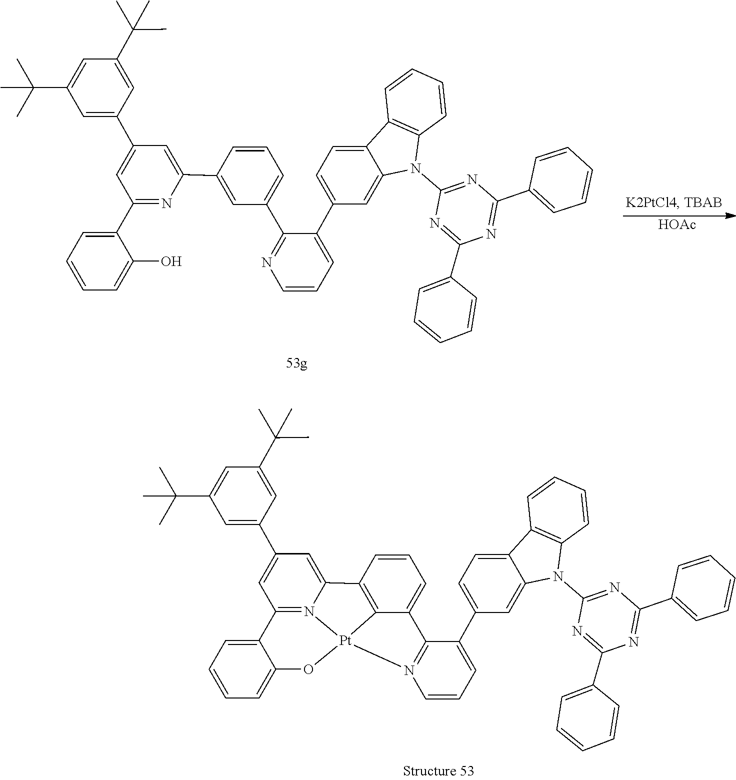

### Synthesis of an Intermediate 53c

53a (14.7 g, 50 mmol), 53b (11.8 g, 50 mmol), toluene (120 mL), ethanol (60 mL), and a 2 mol/L potassium carbonate solution (60 mL) were added into a 500 ml dry double-neck flask, and subjected to ultrasonic treatment for 5-10 min. Then, nitrogen was rapidly stirred for 5 min, tetra(triphenylphosphine)palladium (1.8 g, 1.5 mmol) as a catalyst was rapidly added, and a large amount of the nitrogen was introduced for 10 min. A resulting mixture was heated to 100° C., and stirred for 12 h. Then, during treatment, extraction was conducted first, spin-drying was conducted, and column chromatography was conducted with petroleum ether and dichloromethane to obtain 14.5 g of a white solid product with a yield of 90%. Hydrogen spectrum data are as follows:

¹H NMR (400 MHz, Chloroform-d) δ9.72 (s, 1H), 8.37 (dd, J=4.0, 2.2 Hz, 1H), 8.19-8.13 (m, 1H), 8.13-8.06 (m, 1H), 7.91 (dd, J=7.4, 2.3 Hz, 1H), 7.83 (d, J=1.9 Hz, 1H), 7.71 (dd, J=8.1, 2.0 Hz, 1H), 7.55-7.42 (m, 2H), 7.34 (td, J=7.4, 1.3 Hz, 1H), 7.24 (ddd, J=7.9, 7.3, 1.6 Hz, 1H).

### Synthesis of an Intermediate 53d

The 53c (14.5 g, 45 mmol), 1a (28.7 g, 50 mmol), toluene (120 mL), ethanol (60 mL), and a 2 mol/L potassium carbonate solution (60 mL) were added into a 500 ml dry double-neck flask, and subjected to ultrasonic treatment for 5-10 min. Then, nitrogen was rapidly stirred for 5 min, tetra(triphenylphosphine)palladium (1.8 g, 1.5 mmol) as a catalyst was rapidly added, and a large amount of the nitrogen was introduced for 10 min. A resulting mixture was heated to 100° C., and stirred for 12 h. Then, during treatment, extraction was conducted first, spin-drying was conducted, and column chromatography was conducted with petroleum ether and dichloromethane to obtain g of a white solid product with a yield of 78%. Hydrogen spectrum data are as follows:

¹H NMR (400 MHz, Chloroform-d) δ9.72 (s, 1H), 8.71 (dd, J=4.1, 2.2 Hz, 1H), 8.22-8.06 (m, 5H), 7.97-7.86 (m, 5H), 7.84-7.74 (m, 2H), 7.71-7.60 (m, 2H), 7.55-7.47 (m, 2H), 7.45-7.30 (m, 6H), 7.24 (ddd, J=7.9, 7.3, 1.6 Hz, 1H), 7.15 (ddd, J=8.7, 7.5, 1.2 Hz, 1H), 6.90 (dd, J=7.7, 1.2 Hz, 1H), 3.90 (s, 3H), 1.35 (s, 18H).

### Synthesis of an Intermediate 53f

The 53d (5.0 g, 1.0 eq), 53e (7.0 g, 3.0 eq), Cu (230 mg, 0.5 eq), CuI (688 mg, 0.5 eq), 1,10-phenanthroline (1.30 g, 1.0 eq), and cesium carbonate (7.06 g, 3.0 eq) were added into a 250 ml three-mouth flask, and subjected to a reaction at an oil bath temperature of 160° C. for 3 d under the protection of nitrogen with 100 ml of anhydrous xylene as a reaction solvent. After the reaction was completed, cooling was conducted to room temperature. Then, a resulting reaction solution was directly subjected to suction filtration with EA as an eluent to remove an inorganic salt, and chromatography was conducted with a silica gel column (with a mixture of hex and EA at a ratio of 8:1 as a chromatography solution) to obtain 4.1 g of a yellow fluorescent product point by collection. Hydrogen spectrum data are as follows: ¹H NMR (400 MHz, Chloroform-d) δ8.71 (dd, J=4.1, 2.2 Hz, 1H), 8.40-8.28 (m, 5H), 8.28-8.16 (m, 3H), 8.11 (d, J=2.2 Hz, 1H), 7.97-7.85 (m, 5H), 7.85-7.77 (m, 4H), 7.67 (t, J=8.5 Hz, 1H), 7.57-7.35 (m, 14H), 7.31 (ddd, J=7.9, 7.2, 1.6 Hz, 1H), 7.15 (ddd, J=8.7, 7.5, 1.2 Hz, 1H), 6.90 (dd, J=7.7, 1.2 Hz, 1H), 3.90 (s, 3H), 1.35 (s, 18H).

### Synthesis of an Intermediate 53g

The 53f (3.40 g), pyridine hydrochloride (30.0 g), and o-dichlorobenzene (3.0 ml) were added into a 100 ml one-mouth flask, and subjected to a reaction at an oil bath temperature of 200° C. for 8 h under the protection of nitrogen. After the reaction was completed, cooling was conducted to room temperature. Dissolution was conducted with a large amount of water, and extraction was conducted with DCM for three times. Then, an organic phase was spin-dried and subjected to chromatography with a silica gel column (with a mixture of hex and EA at a ratio of 10:1 as a chromatography solution) to obtain 2.9 g of a light yellow product. Hydrogen spectrum data are as follows: ¹H NMR (400 MHz, Chloroform-d) δ8.71 (dd, J=4.1, 2.2 Hz, 1H), 8.40-8.28 (m, 5H), 8.28-8.16 (m, 4H), 8.11 (d, J=2.2 Hz, 1H), 7.99 (dd, J=8.7, 1.2 Hz, 1H), 7.96-7.73 (m, 8H), 7.67 (t, J=8.5 Hz, 1H), 7.56-7.37 (m, 13H), 7.36-7.20 (m, 2H), 7.03-6.94 (m, 2H), 1.35 (s, 18H).

### Synthesis of an End-Product Compound Having a Structure 53

The 53g (115 mg, 0.124 mmol), K₂PtCl₄ (70 mg, 0.167 mmol), 18-crown-6-ether (6 mg, 0.012 mmol), and acetic acid (5 mL) were put into a 250 ml one-mouth flask, and subjected to a reaction at 130 ° C. for 48 h under the protection of nitrogen. After the reaction was completed, an excessive amount of deionized water was added to precipitate out a solid. Then suction filtration was conducted, and a solid was dissolved in dichloromethane, spin-dried, and subjected to treatment with a silica gel column (with a mixture of Hex, DCM and EA at a ratio of 20:20:1). After the treatment with the column, a resulting product was subjected to recrystallization with a mixture of dichloromethane and n-hexane at a ratio of 1:4. 85 mg of a red solid was obtained. Hydrogen spectrum data are as follows: ¹H NMR (400 MHz, Chloroform-d) δ8.84 (dd, J=6.0, 1.8 Hz, 1H), 8.40-8.28 (m, 5H), 8.28-8.18 (m, 2H), 8.15-8.10 (m, 1H), 8.04-7.99 (m, 1H), 7.94 (dd, J=8.1, 1.2 Hz, 1H), 7.80-7.61 (m, 7H), 7.59-7.42 (m, 13H), 7.39-7.26 (m, 5H), 7.25-7.15 (m, 2H), 7.09 (ddd, J=8.5, 7.5, 1.2 Hz, 1H), 1.35 (s, 18H).

### Luminous Properties of Compounds

| | | Emission |
|---|---|---|
| | | spectrum |
| | Absorption | (dichloromethane |
| | spectrum | solution) |
| Complex | λ/nm | λ/nm |
| Structure 1 | 237.5, 286.6, 357.4 | 518 |
| Structure 26 | 243, 285.5, 381.3 | 517 |
| Structure 36 | 242, 286.9, 383 | 520 |
| Structure 53 | 244, 286.6, 385 | 528 |

An application example of the compound of the present invention is described below.

A preparation method of a device is as follows.

First, a transparent conductive ITO glass substrate 10 (with an anode 20 on the surface) was sequentially washed with a detergent solution, deionized water, ethanol, acetone and deionized water, and then treated with oxygen plasma for 300 s.

Then, HATCN was evaporated on the ITO to serve as a hole injection layer 30 having a thickness of 3 nm.

Then, a TAPC compound was evaporated to form a hole transport layer 40 having a thickness of 50 nm.

Then, a guest complex (9%) and host TCTA (91%) were evaporated on the hole transport layer to serve as a light-emitting layer 50 having a thickness of 7 nm.

Then, a guest complex (9%) and host TCTA (91%) were evaporated on the hole transport layer to serve as a light-emitting layer 60 having a thickness of 3 nm.

Then, TmPyPb was evaporated on the light-emitting layer to serve as a hole blocking layer 70 having a thickness of 50 nm.

Finally, LiF was evaporated to serve as an electron injection layer 80 having a thickness of 0.8 nm, and Al was evaporated to serve as a device cathode 80 having a thickness of 100 nm.

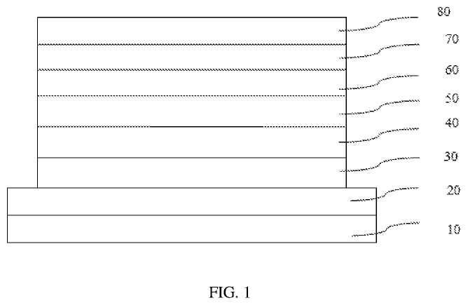

The structure of the device obtained is as shown in FIG. 1.

Structural formulas of the compounds used in the device are as follows.

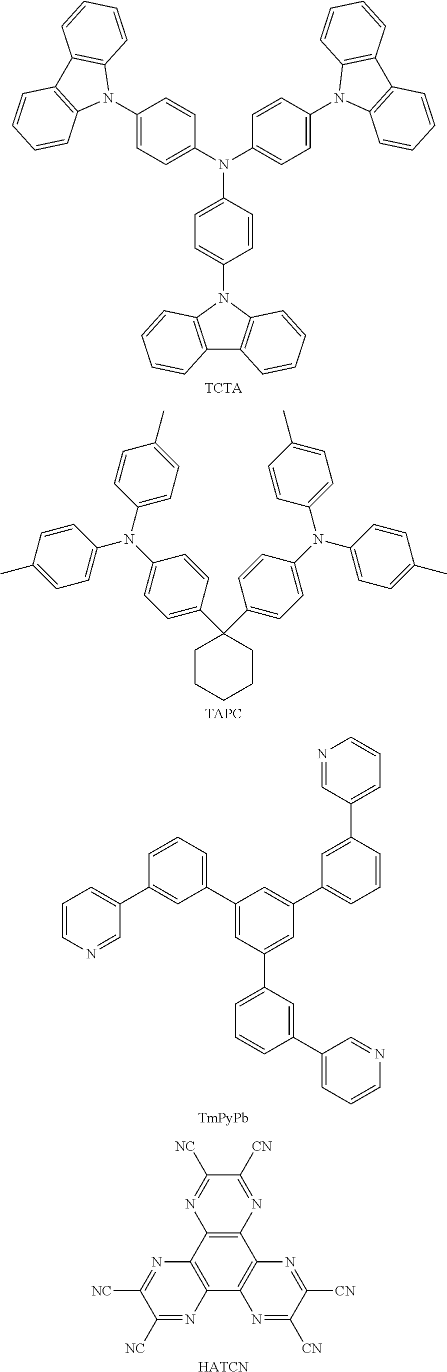

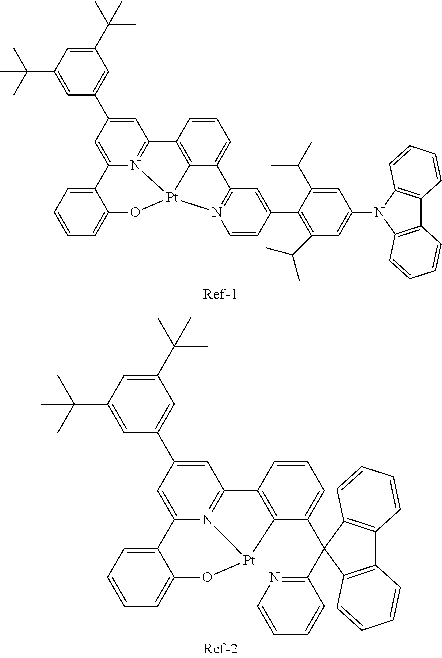

Device results are as follows.

Device properties of organic electroluminescent devices in Comparative Example 1 and Comparative Example 2 at a current density of 20 mA/cm² are listed in Table 1.

TABLE 1

| | | | | Device |
|---|---|---|---|---|
| | | | | service |
| Device | | Driving | Luminous | life |
| number | Complex | voltage | efficiency | (LT95) |
| Comparative | Ref-1 | 1.1 | 0.91 | 0.30 |
| Example 1 | | | | |
| Comparative | Ref-2 | 1.1 | 0.87 | 0.18 |
| Example 2 | | | | |
| Example 5 | Structure 1 | 1 | 0.90 | 0.56 |
| Example 6 | Structure 26 | 1 | 1 | 0.8 |
| Example 7 | Structure 36 | 1 | 1 | 1 |
| Example 8 | Structure 53 | 0.9 | 1.06 | 1.1 |

Note:
A device property test is carried out on the basis of Example 7, and each index is set as 1; and LT95 indicates the corresponding time when the brightness of a device is reduced to 95% of the initial brightness (10,000 cd/m²).

The organic metal complex in the present invention maintains good high quantum efficiency, slightly reduces the driving voltage of a device, and improves the luminous efficiency. However, compared with the LT95 in comparative examples, the device service life in examples is significantly and essentially prolonged. Data of the series of devices show that when the bivalent platinum complex is used as a phosphorescent luminescent ligand material, an OLED device having high luminous efficiency can be manufactured and have an extremely long service life.

The various embodiments described above are merely used as examples, and are not intended to limit the scope of the present invention. On the premise of not departing from the spirit of the present invention, a variety of materials and structures in the present invention can be replaced with other materials and structures. It shall be understood that many modifications and changes can be made by a person skilled in the art without creative effort according to the concept of the present invention. Therefore, all technical solutions that can be obtained by a person skilled in the art through analysis, reasoning or partial research on the basis of the prior art shall fall within the protection scope as defined by this application.

1. A bivalent platinum complex, having a structural formula as shown in a formula (I): 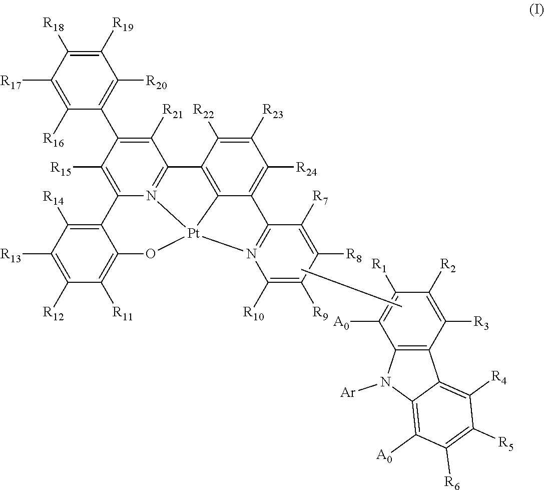
wherein one site of R₁-R₆ in R₁-R₂₄ is connected to one site of R₇-R₁₀ by a C—C bond, remaining sites and A₀ are independently selected from hydrogen, deuterium, halogen, substituted or unsubstituted alkyl containing 1-20 carbon atoms, substituted or unsubstituted cycloalkyl containing 3-20 ring carbon atoms, substituted or unsubstituted heteroalkyl containing 1-20 carbon atoms, substituted or unsubstituted aralkyl containing 7-30 carbon atoms, substituted or unsubstituted alkoxyl containing 1-20 carbon atoms, substituted or unsubstituted aryloxyl containing 6-30 carbon atoms, substituted or unsubstituted alkenyl containing 2-20 carbon atoms, substituted or unsubstituted aryl containing 6-30 carbon atoms, substituted or unsubstituted heteroaryl containing 3-30 carbon atoms, substituted or unsubstituted alkylsilyl containing 3-20 carbon atoms, substituted or unsubstituted arylsilyl containing 6-20 carbon atoms, substituted or unsubstituted amino containing 0-20 carbon atoms, acyl, carbonyl, carboxyl, ester group, cyano, thio, sulfinyl, sulfonyl, and phosphino, or any adjacent two of the R₁-R₂₄ are connected to each other through a covalent bond to form a ring;
Ar is selected from substituted or unsubstituted alkyl containing 1-20 carbon atoms, substituted or unsubstituted cycloalkyl containing 3-20 ring carbon atoms, substituted or unsubstituted heteroalkyl containing 1-20 carbon atoms, substituted or unsubstituted aralkyl containing 7-30 carbon atoms, substituted or unsubstituted alkoxyl containing 1-20 carbon atoms, substituted or unsubstituted aryloxyl containing 6-30 carbon atoms, substituted or unsubstituted alkenyl containing 2-20 carbon atoms, substituted or unsubstituted aryl containing 6-30 carbon atoms, substituted or unsubstituted heteroaryl containing 3-30 carbon atoms, substituted or unsubstituted alkylsilyl containing 3-20 carbon atoms, and substituted or unsubstituted arylsilyl containing 6-20 carbon atoms;
the heteroalkyl or the heteroaryl comprises an N, S, or O heteroatom;
and the “substituted” refers to substitution with deuterium, halogen, amino, nitro, cyano, or C₁-C₄ alkyl.

2. The bivalent platinum complex according to claim 1, wherein one site of the R₁-R₆ in the R₁-R₂₄ is connected to one site of the R₇-R₁₀ by a C—C bond, the remaining sites and the A₀ are independently selected from hydrogen, deuterium, halogen, alkoxyl containing 1-10 C atoms, cyano, styrenyl, aryloxyl, diarylamino, saturated alkyl containing 1-10 C atoms, unsaturated alkyl containing 2-8 C atoms, substituted or unsubstituted aryl containing 5-20 C atoms, and substituted or unsubstituted heteroaryl containing 5-20 C atoms, or any adjacent two of the R₁-R₂₄ are connected to each other through a covalent bond to form a ring.

3. The bivalent platinum complex according to claim 2, wherein the Ar is selected from a substituted or unsubstituted o-aryl derivative or o-heteroaryl derivative containing 6-30 carbon atoms, and a substituted or unsubstituted biaryl derivative or biheteroaryl derivative containing 3-30 carbon atoms.

4. The bivalent platinum complex according to claim 3, wherein one site of the R₁-R₆ in the R₁-R₂₄ is connected to one site of the R₇-R₁₀ by a C—C bond, the remaining sites are hydrogen except that R₁₇ and R₁₉ are a C₁-C₄ group, and the A₀ is hydrogen.

5. The bivalent platinum complex according to claim 4, wherein the Ar is selected from substituted or unsubstituted aryl containing 5-30 carbon atoms, heteroaryl, and benzoheteroaryl.

6. The bivalent platinum complex according to claim 5, wherein the R₁₇ and the R₁₉ are isobutyl; the Ar is selected from substituted or unsubstituted five-membered or six-membered heteroaryl containing phenyl, and benzoheteroaryl; the heteroaryl comprises an N or O heteroatom; and the “substituted” refers to substitution with deuterium, halogen, or C₁-C₄ alkyl.

7. The bivalent platinum complex according to claim 1, having one of the following structures:

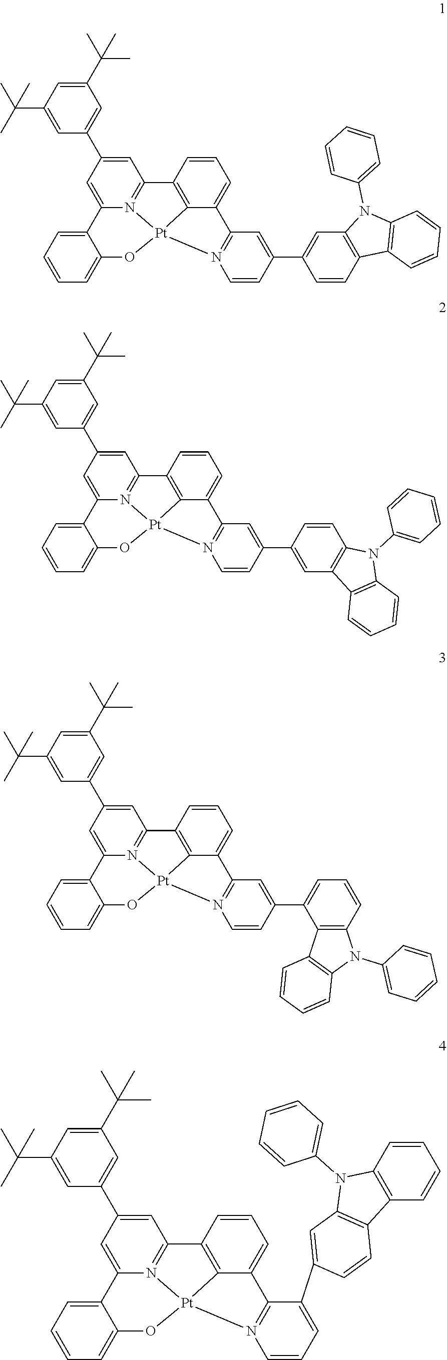

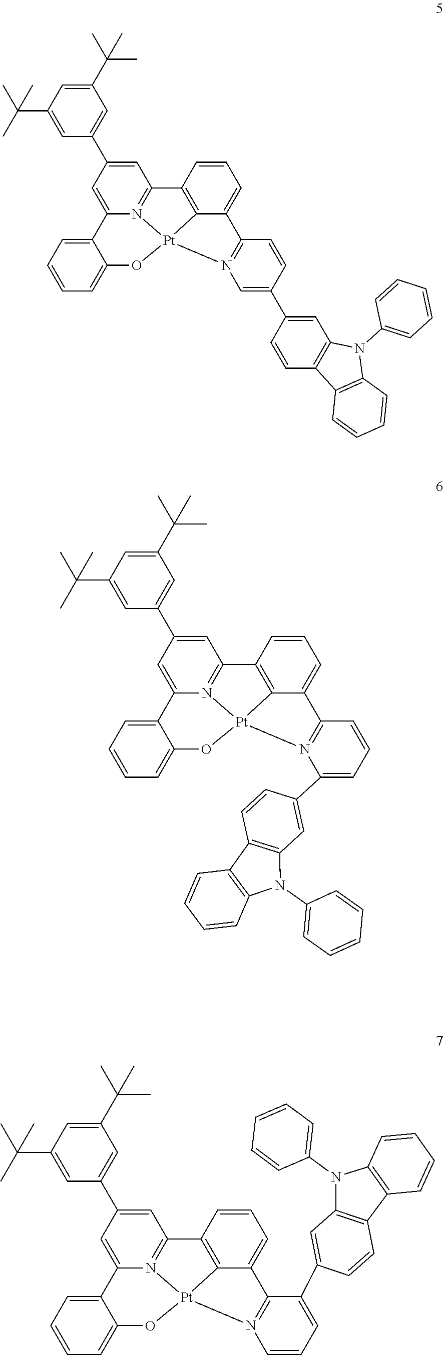

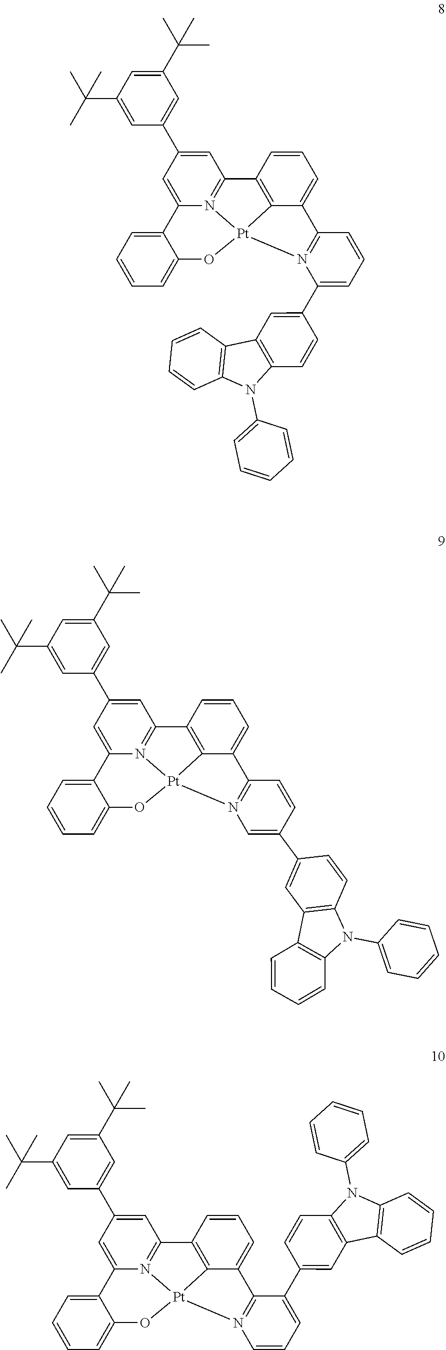

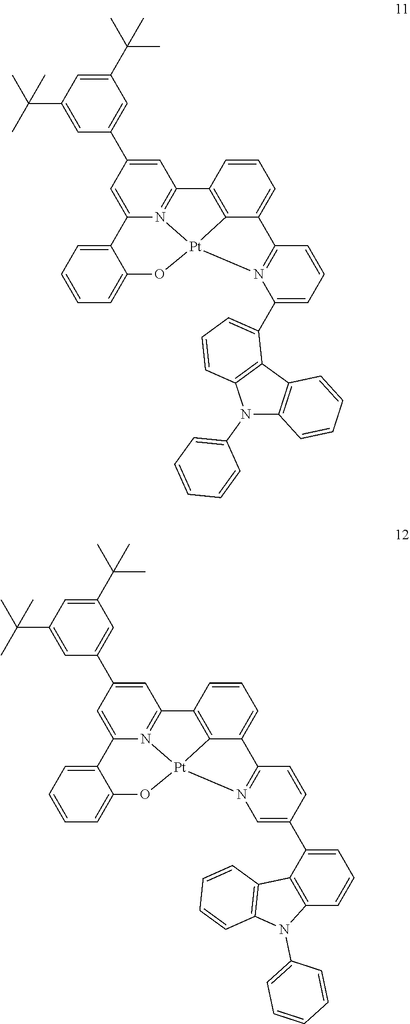

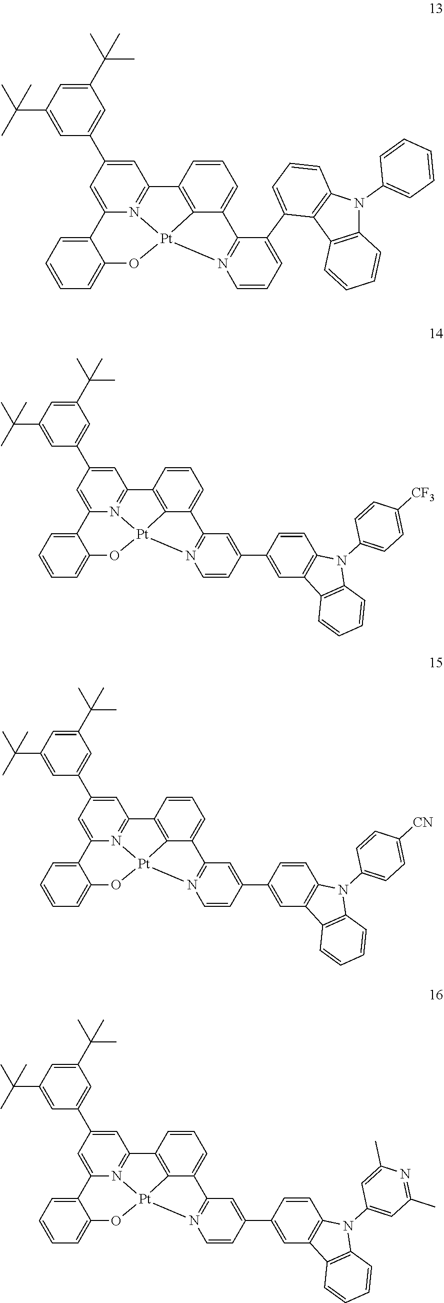

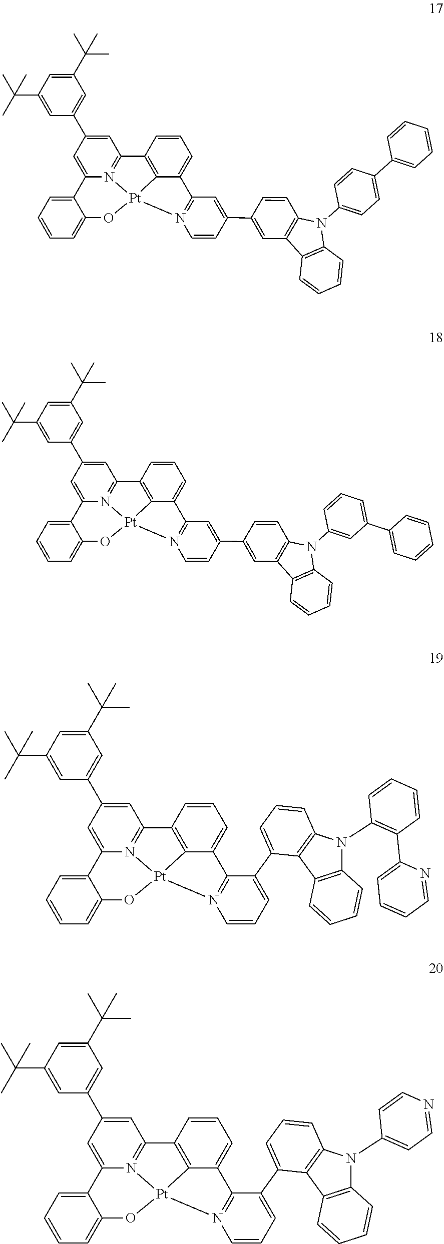

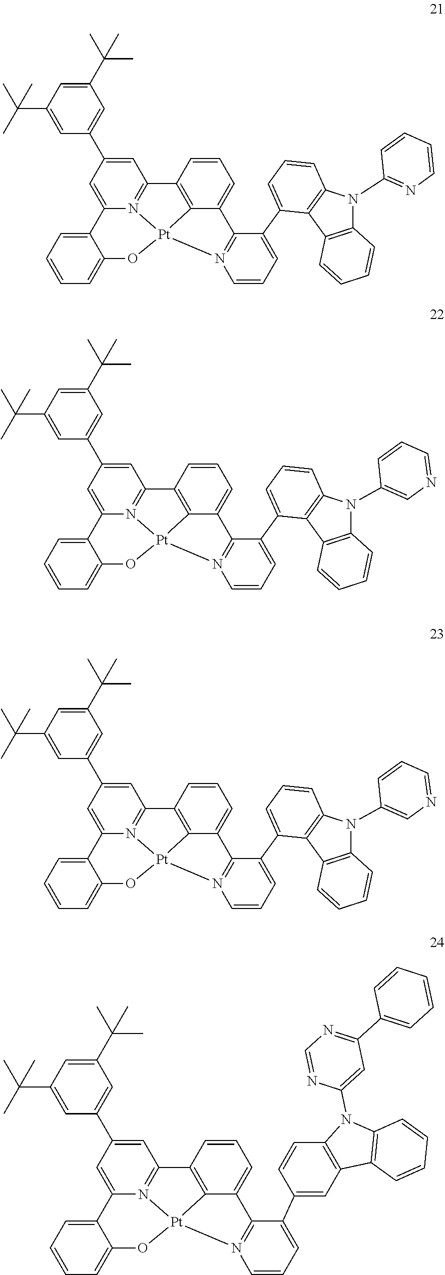

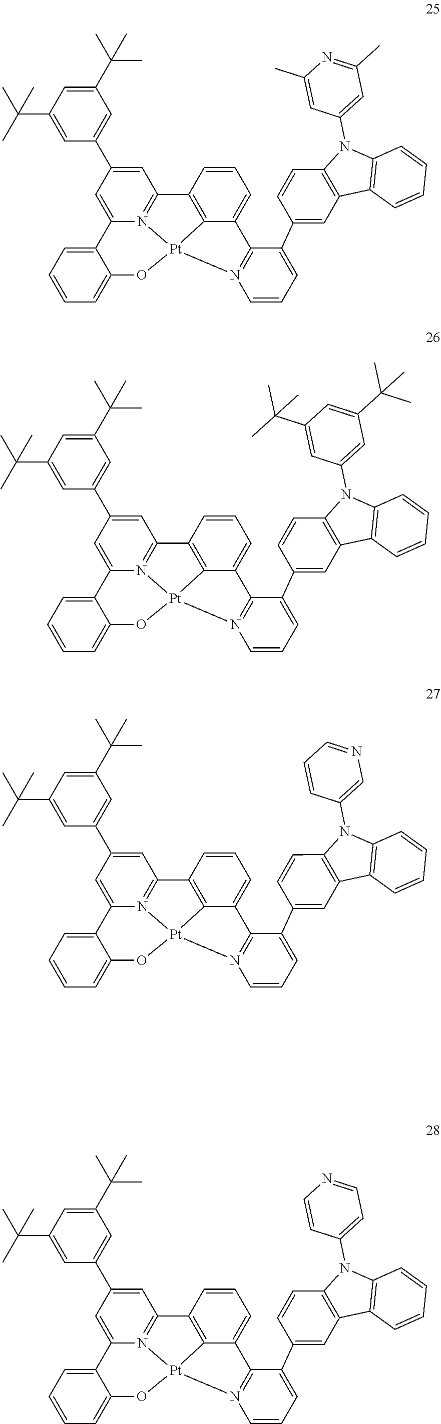

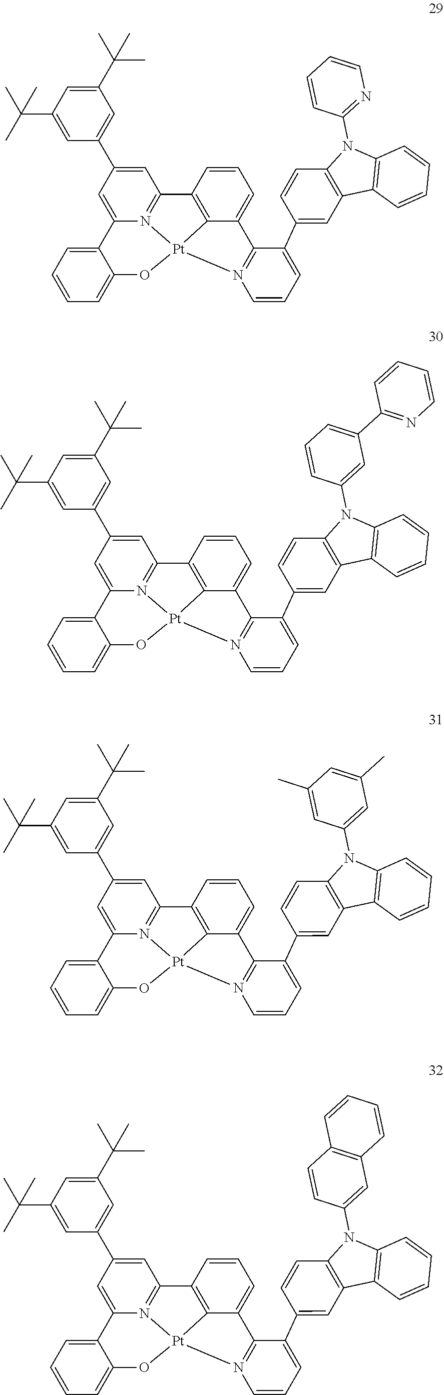

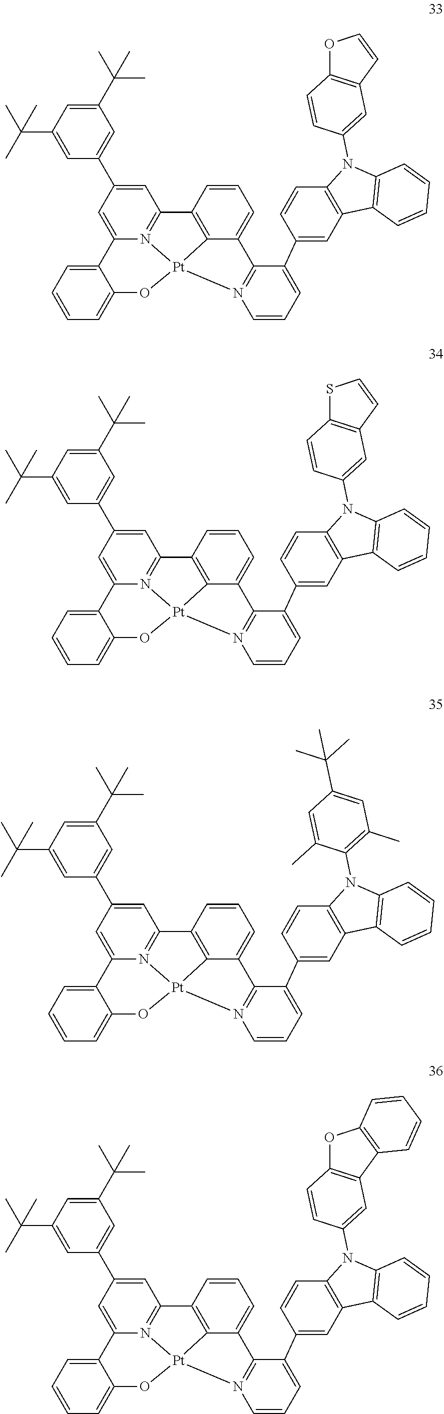

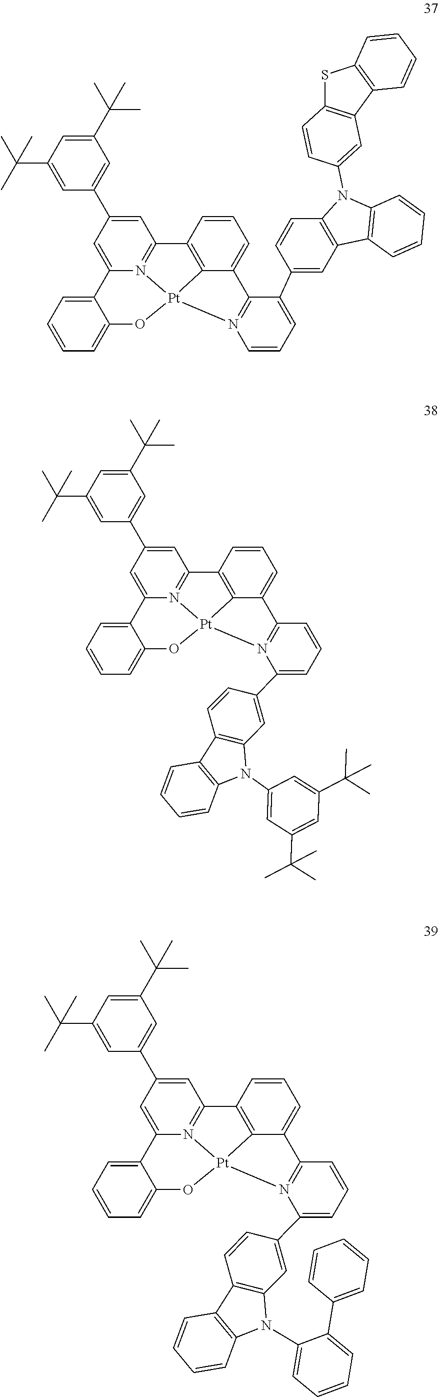

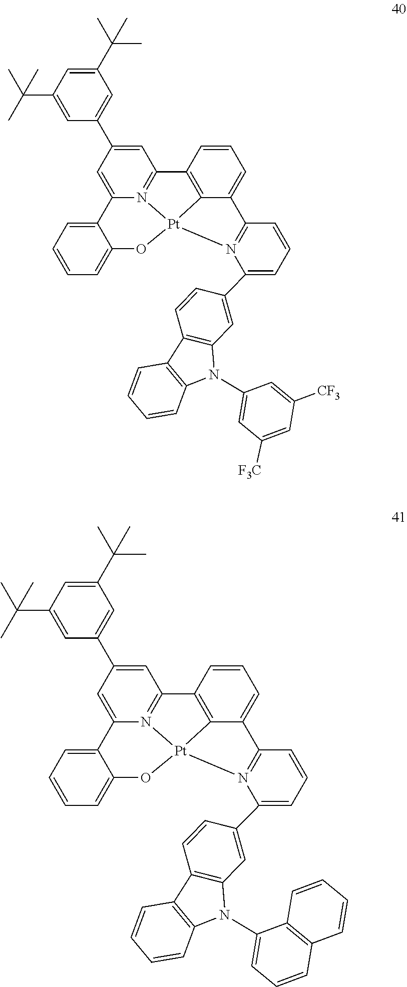

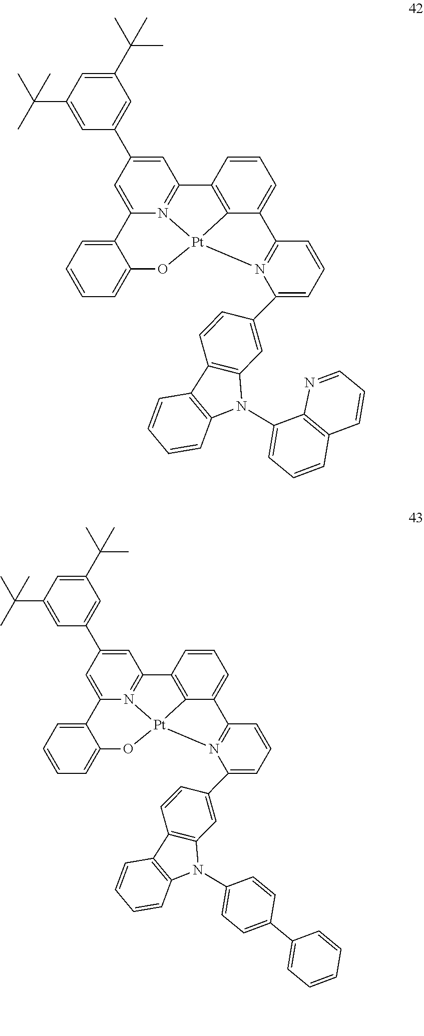

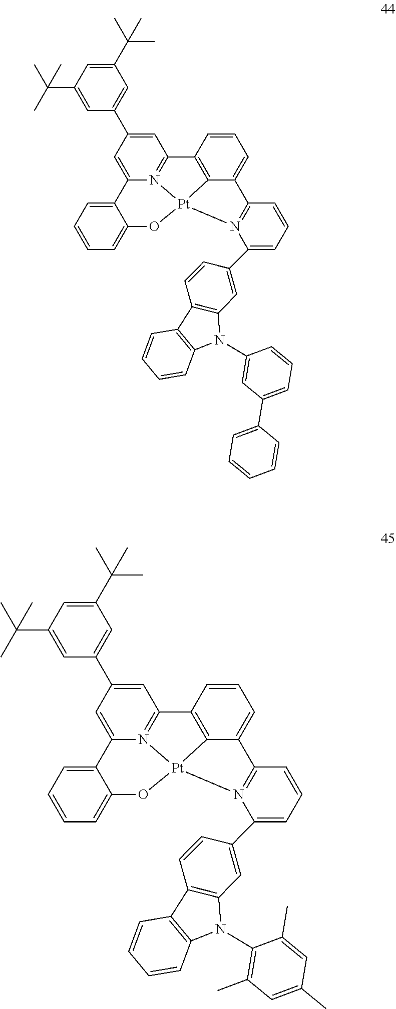

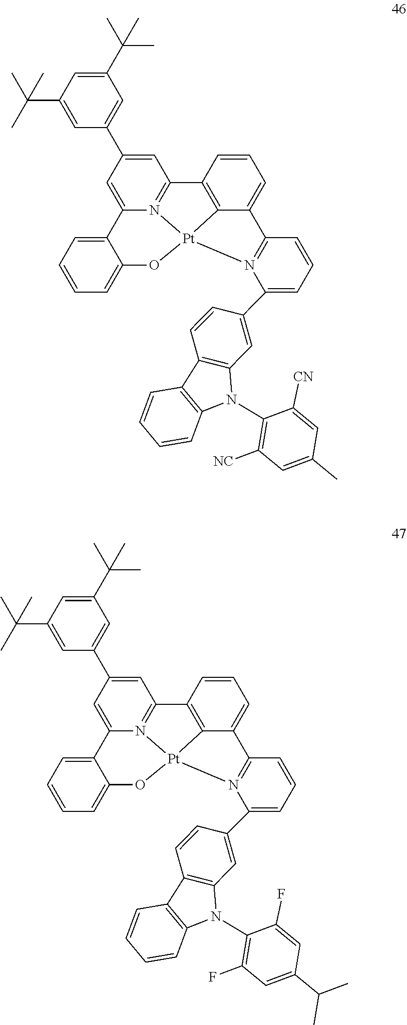

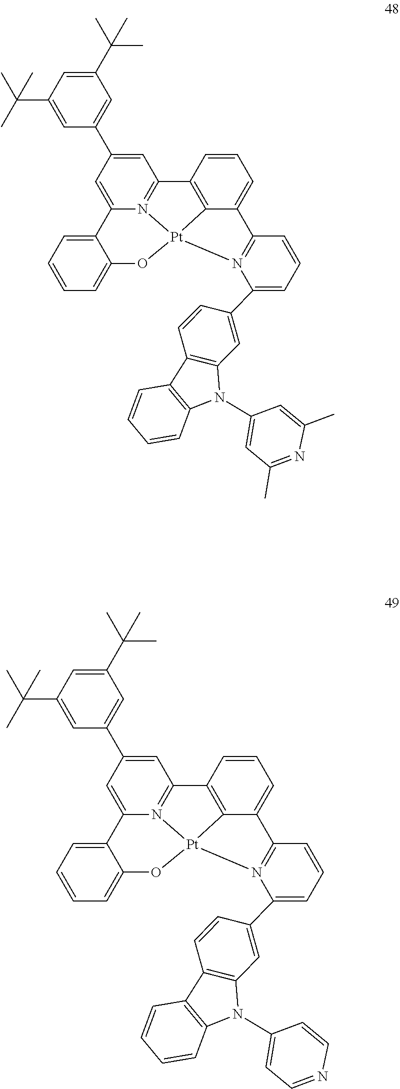

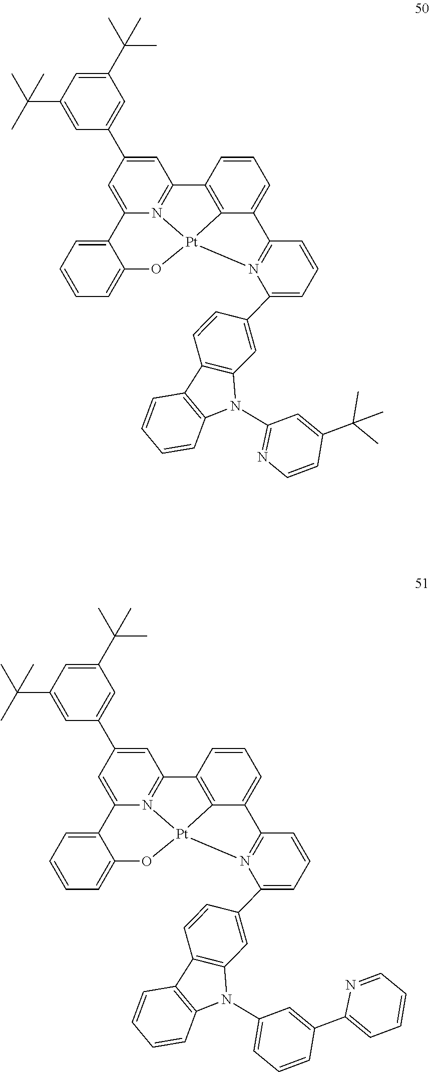

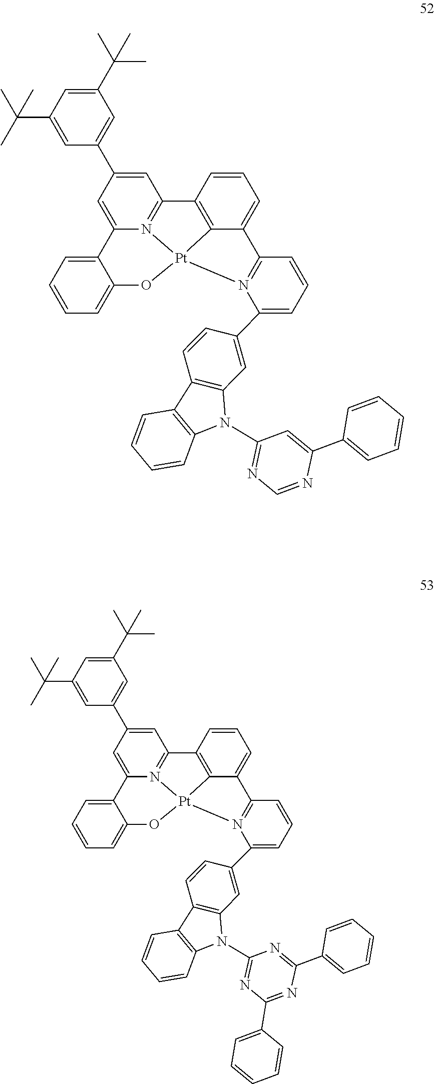

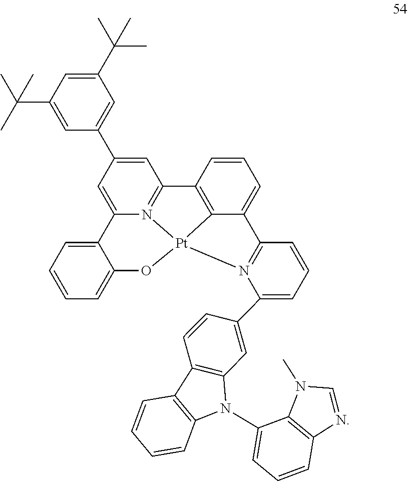

8. A precursor of the bivalent platinum complex according to claim 1, having a structure as shown in the following formula: 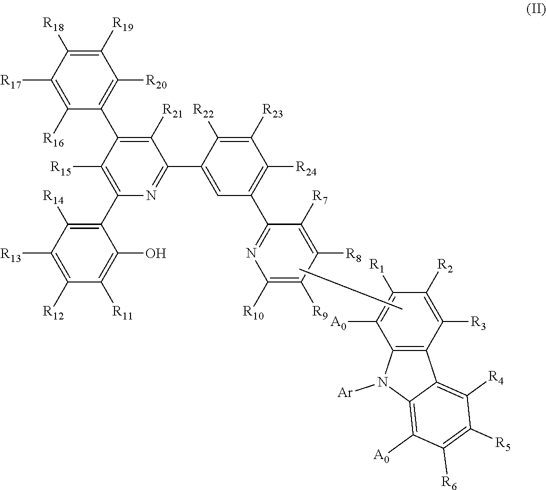
wherein R₁-R₂₄, A₀ and Ar are defined the same as above.

9. Application of the bivalent platinum complex according to claim 1 as a phosphorescent doping material for a light-emitting layer in an OLED.

10. A precursor of the bivalent platinum complex according to claim 2, having a structure as shown in the following formula: 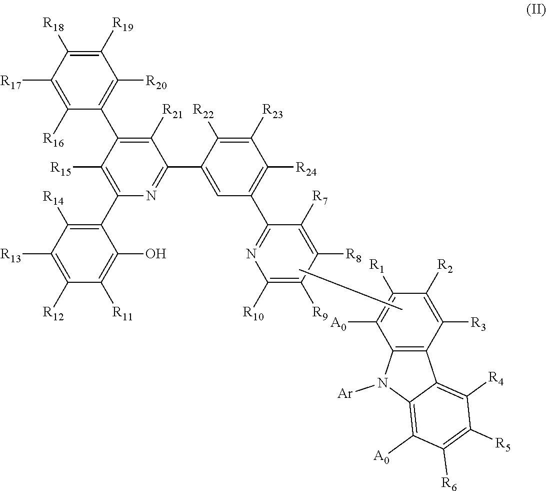
wherein R₁-R₂₄, A₀ and Ar are defined the same as above.

11. A precursor of the bivalent platinum complex according to claim 3, having a structure as shown in the following formula: 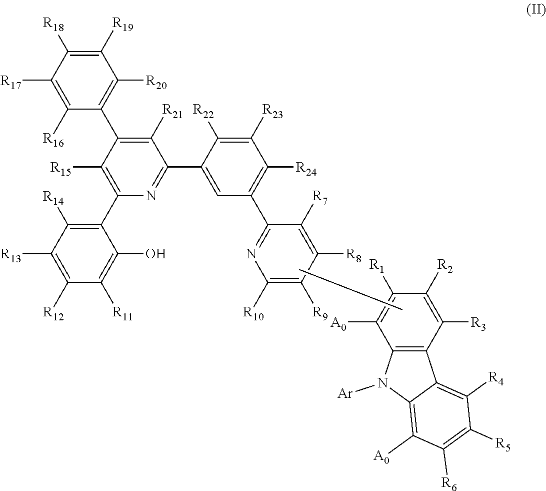
wherein R₁-R₂₄, A₀ and Ar are defined the same as above.

12. A precursor of the bivalent platinum complex according to claim 4, having a structure as shown in the following formula: 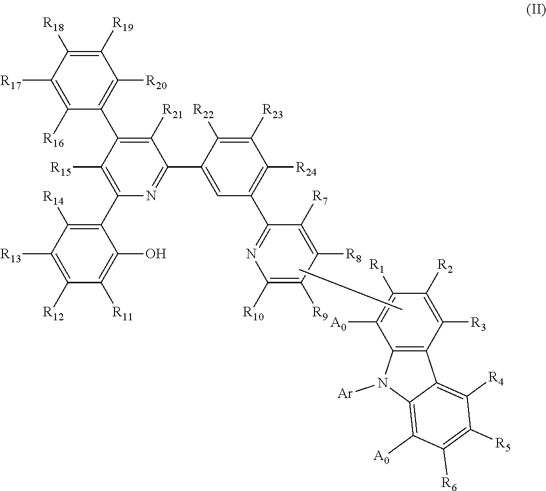
wherein R₁-R₂₄, A₀ and Ar are defined the same as above.

13. A precursor of the bivalent platinum complex according to claim 5, having a structure as shown in the following formula: 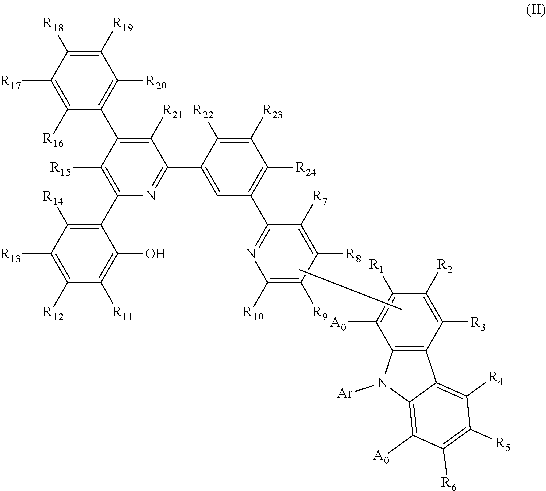
wherein R₁-R₂₄, A₀ and Ar are defined the same as above.

14. A precursor of the bivalent platinum complex according to claim 6, having a structure as shown in the following formula: 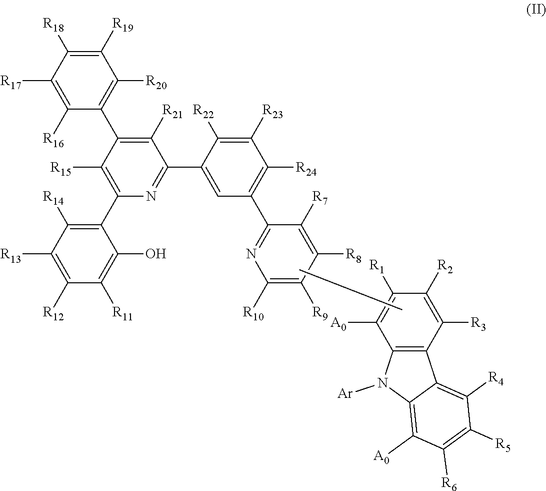
wherein R₁-R₂₄, A₀ and Ar are defined the same as above.

15. A precursor of the bivalent platinum complex according to claim 7, having a structure as shown in the following formula: 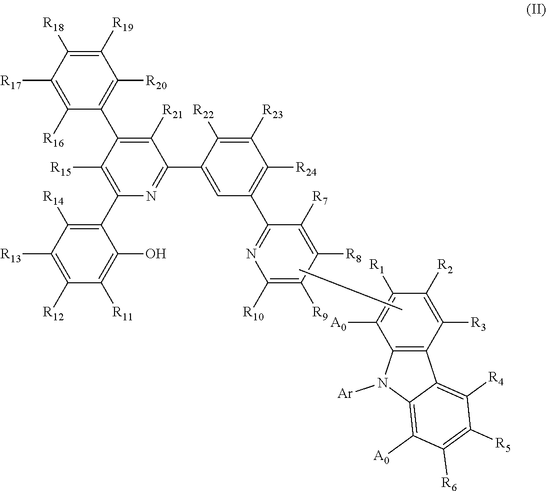
wherein R₁-R₂₄, A₀ and Ar are defined the same as above.

16. Application of the bivalent platinum complex according to claim 2 as a phosphorescent doping material for a light-emitting layer in an OLED.

17. Application of the bivalent platinum complex according to claim 3 as a phosphorescent doping material for a light-emitting layer in an OLED.

18. Application of the bivalent platinum complex according to claim 4 as a phosphorescent doping material for a light-emitting layer in an OLED.

19. Application of the bivalent platinum complex according to claim 5 as a phosphorescent doping material for a light-emitting layer in an OLED.

20. Application of the bivalent platinum complex according to claim 6 as a phosphorescent doping material for a light-emitting layer in an OLED.
